(19) United States
(12) Patent Application Publication
(10) Pub. No.: US 2009/0102373 A1
(43) Pub. Date: Apr. 23, 2009

(54) Electroluminescent device

(76) Inventors: Pascal Hayoz, Hofstetten (CH); Thomas Schafer, Basel (CH); Kristina Bardon, Waldshut (DE)

Correspondence Address: JoAnn Villamizar;Ciba Corporation/Patent Department, 540 White Plains Road, P.O. Box 2005, Tarrytown, NY, 10591, US

(21) Appl. No.: 12/316,720

(22) Filed: Dec. 16, 2008

Related U.S. Application Data
(63) Continuation of application No. 10/546,683, filed on Aug. 23, 2005, now abandoned.

(30) Foreign Application Priority Data
Feb. 28, 2003 (EP) 03100501.0
Jul. 30, 2003 (EP) 03102360.9

(51) Int. Cl. H01J 1/63; C07D 251/14

(52) U.S. Cl. 313/504; 544/180

(57) ABSTRACT

Disclosed are electroluminescent devices that comprise organic layers that contain triazine compounds. The triazine compounds are suitable components of blue-emitting, durable, organo-electroluminescent layers. The electroluminescent devices may be employed for full color display panels in, for example, mobile phones, televisions and personal computer screens.

This is a continuation of U.S. application Ser. No. 10/546,683, pending, which is a 371 of PCT/EP04/050146, filed Feb. 18, 2004, which are hereby incorporated by reference.

The present invention relates to organo-electroluminescent (EL) devices, in particular EL devices that comprise durable, blue-emitting organo-electroluminescent layers. The organo-electroluminescent layers comprise certain triazine. The present invention is aimed at an electroluminescent device comprising an organic light-emitting layer that contains at least one blue-emitting triazine compound.

U.S. Pat. No. 6,352,791 relates to an electroluminescent arrangement, comprising at least two electrodes, and a light emitting layer system including at least one emitter layer and at least one electron-conducting layer, wherein the at least one electron-conducting layer does not emit light and includes one triazine compound, such as, for example,

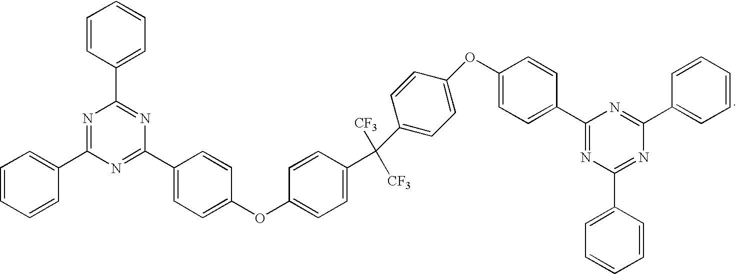

U.S. Pat. No. 6,225,467 is directed to organic electroluminescent (EL) devices, which contain an electron transport component comprised of triazine compounds, such as, for example, 4,6-tris(4-biphenylyl)-1,3,5-triazine, 2,4,6-tris[4-(4′-methylbiphenylyl)]-1,3,5-triazine, 2,4,6-tris[4-(4′-tert-butylbiphenylyl)]-1,3,5-triazine, 2,4,6-tris[4-(3′,′-dimethylbiphenylyl)]-1, 3,5-triazine, 2,4,6-tris[4-(4′-methoxybiphenylyl)]-1,3,5-triazine, 2,4,6-tris[4-(3′-methoxybiphenylyl)]-1,3,5-triazine, 2,4-bis(4-biphenylyl)-6-phenyl-1,3,5-triazine and 2,4-bis(4-biphenylyl)-6-m-tolyl-1,3,5-triazine.

EP-A-1,202,608 relates to an electroluminescent arrangement, wherein a host material constituting the hole transporting layer is a compound of formula

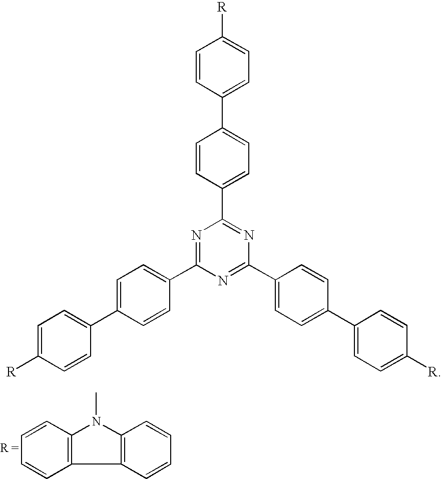

EP-A-1,013,740 relates to an electroluminescent element, wherein among others the following compound can be used as EL material:

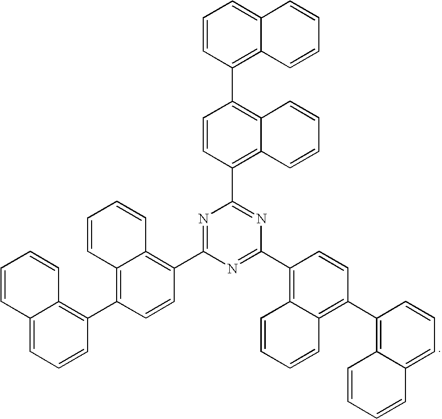

It is the object of the present invention to provide a light emitting element with excellent light emitting characteristics and durability.

Accordingly the present invention relates to an electroluminescent device comprising an anode, a cathode and one or a plurality of organic compound layers sandwiched therebetween, in which said organic compound layers comprise a triazine compound of formula

W is a group of formula

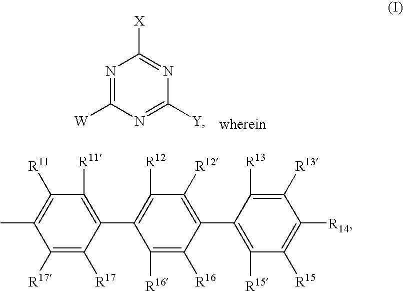

X and Y are independently of each other an aryl group or a heteroaryl group, especially a group of formula

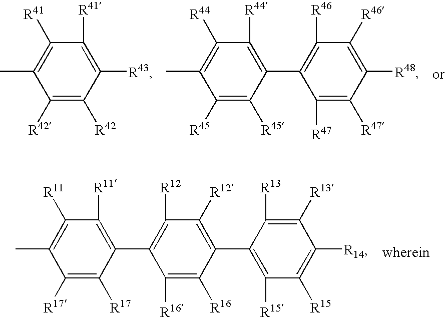

R¹¹, R^{11′}, R¹², R^{12′}, R¹³, R^{13′}, R¹⁵, R^{15′}, R¹⁶, R^{16′}, R¹⁷, R^{17′}, R⁴¹, R^{41′}, R⁴², R^{42′}, R⁴⁴, R^{44′}, R⁴⁵, R^{45′}, R⁴⁶, R^{46′}, R⁴⁷ and R^{47′} are independently of each other H, E, C₆-C₁₈aryl; C₆-C₁₈aryl which is substituted by G; C₁-C₁₈alkyl; C₁-C₁₈alkyl which is substituted by E and/or interrupted by D; C₇-C₁₈aralkyl; or C₇-C₁₈aralkyl which is substituted by G; or
R^{11′} and R¹², R^{12′} and R¹³, R^{15′} and R¹⁶, R^{16′} and R¹⁷, R^{44′} and R⁴⁶ and/or R^{45′} and R⁴⁷ are each a divalent group L¹ selected from an oxygen atom, an sulfur atom, >CR¹⁸R¹⁹>SiR¹⁸R¹⁹, or

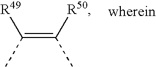

R¹⁸ and R¹⁹ are independently of each other C₁-C₁₈alkyl; C₁-C₁₈alkoxy, C₆-C₁₈aryl; C₇-C₁₈aralkyl;
R¹¹ and R^{11′}, R¹² and R^{12′}, R¹³ and R^{13′}, R¹⁴ and R¹⁴, and R¹⁵, R¹⁵ and R^{15′}, R¹⁶ and R^{16′}, R^{17′} and R¹⁷, R⁴¹ and R^{41′}, R⁴² and R^{42′}, R^{42′} and R⁴³, R^{41′} and R⁴³, R⁴⁴ and R^{44′} R⁴⁵ and R^{45′}, R⁴⁶ and R^{46′}, R⁴⁷ and R^{47′}, R^{46′} and R⁴⁸ and/or R^{47′} and R⁴⁸ are each a divalent group

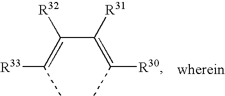

R³⁰, R³¹, R³², R³³, R⁴⁹ and R⁵⁰ are independently of each other H, C₁-C₁₈alkyl; C₁-C₁₈alkyl, which is substituted by E and/or interrupted by D; E; C₆-C₁₈aryl; C₆-C₁₈aryl, which is substituted by E;
R¹⁴ is H, C₂-C₃₀heteroaryl, C₂-C₃₀heteroaryl, which is substituted by G, C₆-C₃₀aryl, or C₆-C₃₀aryl, which is substituted by G, C₁-C₁₈alkyl; or C₁-C₁₈alkyl which is substituted by E and/or interrupted by D; especially

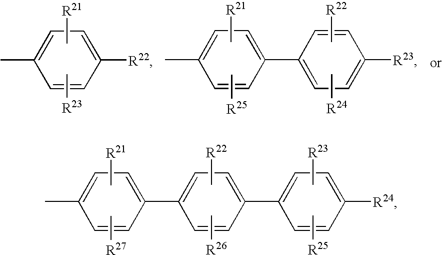

wherein R²¹, R²², R²³, R²⁴, R²⁵, R²⁶ and R²⁷ are independently of each other H, E, C₁-C₁₈alkyl; C₁-C₁₈alkyl which is substituted by E and/or interrupted by D; E; C₇-C₁₈aralkyl; C₇-C₁₈aralkyl which is substituted by G;
R⁴³ and R⁴⁸ are independently of each other H, E; C₁-C₁₈alkyl; C₁-C₁₈alkyl, which is substituted by E and/or interrupted by D; C₂-C₃₀heteroaryl; C₂-C₃₀heteroaryl, which is substituted by G, C₇-C₁₈aralkyl; or C₇-C₁₈aralkyl which is substituted by G;
D is —CO—; —COO—; —OCOO—; —S—; —SO—; —SO₂—; —O—; —NR⁵—; SiR⁶¹R⁶²—; —POR⁵—; —CR⁶³═CR⁶⁴—; or —C═C—;
E is —OR⁵; —SR⁵; —NR⁵R⁶; —COR⁸; —COOR⁷; —OCOOR⁷, —CONR⁵R⁶; —CN; or halogen;
G is E, or C₁-C₁₈alkyl,
wherein R⁵ and R⁶ are independently of each other C₆-C₁₈aryl; C₆-C₁₈aryl which is substituted by C₁-C₁₈alkyl, C₁-C₁₈alkoxy; or C₁-C₁₈alkyl which is interrupted by —O—; or
R⁵ and R⁶ together form a five or six membered ring, in particular

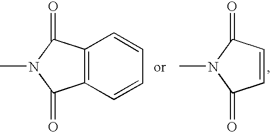

R⁷ is C₆-C₁₈aryl; C₆-C₁₈aryl which is substituted by C₁-C₁₈alkyl, C₁-C₁₈alkoxy; or C₁-C₁₈alkyl which is interrupted by —O—;

R⁸ is C₇-C₁₂alkylaryl; C₁-C₁₈alkyl; or C₁-C₁₈alkyl which is interrupted by —O—;
R⁶¹ and R⁶² are independently of each other C₆-C₁₈aryl; C₆-C₁₈aryl which is substituted by C₁-C₁₈alkyl, C₁-C₁₈alkoxy; or C₁-C₁₈alkyl which is interrupted by —O—, and
R⁶³ and R⁶⁴ are independently of each other H, C₆-C₁₈aryl; C₆-C₁₈aryl which is substituted by C₁-C₁₈alkyl, C₁-C₁₈alkoxy; or C₁-C₁₈alkyl which is interrupted by —O—.

In general, the triazine compound or compounds emit light below about 520 nm, in particular between about 380 nm and about 520 nm.

The triazine compound or compounds have a NTSC coordinate of between about (0.12, 0.05) and about (0.16, 0.10), preferably a NTSC coordinate of about (0.14, 0.08).

The triazine compound or compounds have a melting point above about 150° C., preferably above about 200° C. and most preferred above about 250° C.

To obtain organic layers of this invention with the proper T_{g}, or glass transition temperature, it is advantageous that the present organic compounds have a glass transition temperature greater than about 100° C., for example greater than about 110° C., for example greater than about 120° C., for instance greater than about 130° C.

The electroluminescent devices of the present invention are otherwise designed as is known in the art, for example as described in U.S. Pat. Nos. 5,518,824, 6,225,467, 6,280,859, 5,629,389, 5,486,406, 5,104,740, 5,116,708 and 6,057,048, the relevant disclosures of which are hereby incorporated by reference.

For example, organic EL devices contain one or more layers such as:
substrate; base electrode; hole-injecting layer; hole transporting layer; emitter layer; electron-transporting layer; electron-injecting layer; top electrode; contacts and encapsulation.
This structure is a general case and may have additional layers or may be simplified by omitting layers so that one layer performs a plurality of tasks. For instance, the simplest organic EL device consists of two electrodes which sandwich an organic layer that performs all functions, including the function of light emission.

A preferred EL device comprises in this order:

(a) an anode,
(b) a hole injecting layer and/or a hole transporting layer,
(c) a light-emitting layer,
(d) optionally an electron transporting layer and
(e) a cathode.

In particular, the present organic compounds function as light emitters and are contained in the light emission layer or form the light-emitting layer.

The light emitting compounds of this invention exhibit intense fluorescence in the solid state and have excellent electric-field-applied light emission characteristics. Further, the light emitting compounds of this invention are excellent in the injection of holes from a metal electrode and the transportation of holes; as well as being excellent in the injection of electrons from a metal electrode and the transportation of electrons. They are effectively used as light emitting materials and may be used in combination with other hole transporting materials, other electron transporting materials or other dopants.
The organic compounds of the present invention form uniform thin films. The light emitting layers may therefore be formed of the present organic compounds alone.
Alternatively, the light-emitting layer may contain a known light-emitting material, a known dopant, a known hole transporting material or a known electron transporting material as required. In the organic EL device, a decrease in the brightness and life caused by quenching can be prevented by forming it as a multi-layered structure. The light-emitting material, a dopant, a hole-injecting material and an electron-injecting material may be used in combination as required. Further, a dopant can improve the light emission brightness and the light emission efficiency, and can attain the red or blue light emission. Further, each of the hole transporting zone, the light-emitting layer and the electron transporting zone may have the layer structure of at least two layers. In the hole transporting zone in this case, a layer to which holes are injected from an electrode is called “hole-injecting layer”, and a layer which receives holes from the hole-injecting layer and transport the holes to a light-emitting layer is called “hole transporting layer”. In the electron transporting zone, a layer to which electrons are injected from an electrode is called “electron-injecting layer”, and a layer which receives electrons from the electron-injecting layer and transports the electrons to a light-emitting layer is called “electron transporting layer”. These layers are selected and used depending upon factors such as the energy level and heat resistance of materials and adhesion to an organic layer or metal electrode.
The light-emitting material or the dopant which may be used in the light-emitting layer together with the organic compounds of the present invention includes for example anthracene, naphthalene, phenanthrene, pyrene, tetracene, coronene, chrysene, fluorescein, perylene, phthaloperylene, naphthaloperylene, perinone, phthaloperinone, naphthaloperinone, diphenylbutadiene, tetraphenylbutadiene, coumarine, oxadiazole, aldazine, bisbenzoxazoline, bisstyryl, pyrazine, cyclopentadiene, quinoline metal complex, aminoquinoline metal complex, benzoquinoline metal complex, imine, diphenylethylene, vinyl anthracene, diaminocarbazole, pyran, thiopyran, polymethine, merocyanine, an imidazole-chelated oxynoid compound, quinacridone, rubrene, and fluorescent dyestuffs for a dyestuff laser or for brightening.
The triazine compounds of the present invention and the above compound or compounds that can be used in a light-emitting layer may be used in any mixing ratio for forming a light-emitting layer. That is, the organic compounds of the present invention may provide a main component for forming a light-emitting layer, or they may be a doping material in another main material, depending upon a combination of the above compounds with the organic compounds of the present invention.
The hole-injecting material is selected from compounds which are capable of transporting holes, are capable of receiving holes from the anode, have an excellent effect of injecting holes to a light-emitting layer or a light-emitting material, prevent the movement of excitons generated in a light-emitting layer to an electron-injecting zone or an electron-injecting material and have the excellent capability of forming a thin film. Suitable hole-injecting materials include for example a phthalocyanine derivative, a naphthalocyanine derivative, a porphyrin derivative, oxazole, oxadiazole, triazole, imidazole, imidazolone, imidazolthione, pyrazoline, pyrazolone, tetrahydroimidazole, oxazole, oxadiazole, hydrazone, acylhydrazone, polyarylalkane, stilbene, butadiene, benzidine type triphenylamine, styrylamine type triphenylamine, diamine type triphenylamine, derivatives of these, and polymer materials such as polyvinylcarbazole, polysilane and an electroconducting polymer.

In the organic EL device of the present invention, the hole-injecting material which is more effective is an aromatic tertiary amine derivative or a phthalocyanine derivative. Although not specially limited, specific examples of the tertiary amine derivative include triphenylamine, tritolylamine, tolyldiphenylamine, N,N′-diphenyl-N,N′-(3-methylphenyl)-1,1-biphenyl-4,4′-diamine, N,N,N′,N′-tetra(4-methylphenyl)-1,1′-phenyl-4,4′-diamine, N,N,N′,N′-tetra(4-methylphenyl)-1,1′-biphenyl-4,4′-diamine, N,N′-diphenyl-N,N′-di(1-naphthyl)-1,1′-biphenyl-4,4′-diamine, N,N′-di(methylphenyl)-N,N′-di(4-n-butylphenyl)-phenanthrene-9,10-diamine, 4,4′,4″-tris(3-methylphenyl)-N-phenylamino)triphenylamine, 1,1-bis(4-di-p-tolylaminophenyl)cyclohexane, and oligomers or polymers having aromatic tertiary amine structures of these.

Although not specially limited, specific examples of the phthalocyanine (Pc) derivative include phthalocyanine derivatives or naphthalocyanine derivatives such as H₂Pc, CuPc, CoPc, NiPc, ZnPc, PdPc, FePc, MnPc, ClAlPc, ClGaPc, ClInPc, ClSnPc, Cl₂SiPc, (HO)AlPc, (HO)GaPc, VOPc, TiOPc, MoOPc, and GaPc-O—GaPc.
The hole transporting layer can reduce the driving voltage of the device and improve the confinement of the injected charge recombination within the triazine light emitting layer. Any conventional suitable aromatic amine hole transporting material described for the hole-injecting layer may be selected for forming this layer.

A preferred class of hole transporting materials is comprised of 4,4′-bis(9-carbazolyl)-1,1′-biphenyl compounds of the formula

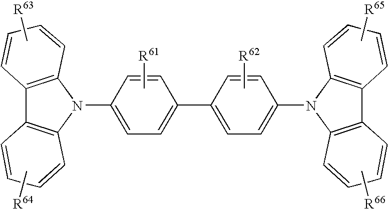

wherein R⁶¹ and R⁶² is a hydrogen atom or an C₁-C₃alkyl group; R⁶³ through R⁶⁶ are substituents independently selected from the group consisting of hydrogen, a C₁-C₆alkyl group, a C₁-C₆alkoxy group, a halogen atom, a dialkylamino group, a C₆-C₃₀aryl group, and the like. Illustrative examples of 4,4′-bis(9-carbazolyl)-1,1′-biphenyl compounds include 4,4′-bis(9-carbazolyl)-1,1′-biphenyl and 4,4′-bis(3-methyl-9-carbazolyl)-1,1′-biphenyl, and the like. The electron transporting layer is not necessarily required for the present device, but is optionally and preferably used for the primary purpose of improving the electron injection characteristics of the EL devices and the emission uniformity. Illustrative examples of electron transporting compounds, which can be utilized in this layer, include the metal chelates of 8-hydroxyquinoline as disclosed in U.S. Pat. Nos. 4,539,507, 5,151,629, and 5,150,006, the disclosures of which are totally incorporated herein by reference.
Although not specially limited, specific examples of the metal complex compound include lithium 8-hydroxyquinolinate, zinc bis(8-hydroxyquinolinate), copper bis(8-hydroxyquinolinate), manganese bis(8-hydroxyquinolinate), aluminum tris(8-hydroxyquinolinate), aluminum tris(2-methyl-8-hydroxyquinolinate), gallium tris(8-hydroxyquinolinate), beryllium bis(10-hydroxybenzo[h]quinolinate), zinc bis(10-hydroxybenzo[h]quinolinate), chlorogallium bis(2-methyl-8-quinolinate), gallium bis(2-methyl-8-quinolinate)(o-cresolate), aluminum bis(2-methyl-8-quinolinate)(1-naphtholate), gallium bis(2-methyl-8-quinolinate)(2-naphtholate), gallium bis(2-methyl-8-quinolinate)phenolate, zinc bis(o-(2-benzooxazolyl)phenolate), zinc bis(o-(2-benzothiazolyl)phenolate) and zinc bis(o-(2-benzotrizolyl)phenolate). The nitrogen-containing five-membered derivative is preferably an oxazole, thiazole, thiadiazole, or triazole derivative. Although not specially limited, specific examples of the above nitrogen-containing five-membered derivative include 2,5-bis(1-phenyl)-1,3,4-oxazole, 1,4-bis(2-(4-methyl-5-phenyloxazolyl)benzene, 2,5-bis(1-phenyl)-1,3,4-thiazole, 2,5-bis(1-phenyl)-1,3,4-oxadiazole, 2-(4′-tert-butylphenyl)-5-(4″-biphenyl)1,3,4-oxadiazole, 2,5-bis(1-naphthyl)-1,3,4-oxadiazole, 1,4-bis[2-(5-phenyloxadiazolyl)]benzene, 1,4-bis[2-(5-phenyloxadiazolyl)-4-tert-butylbenzene], 2-(4′-tert-butylphenyl)-5-(4″-biphenyl)-1,3,4-thiadiazole, 2,5-bis(1-naphthyl)-1,3,4-thiadiazole, 1,4-bis[2-(5-phenylthiazolyl)]benzene, 2-(4′-tert-butylphenyl)-5-(4″-biphenyl)-1,3,4-triazole, 2,5-bis(1-naphthyl)-1,3,4-triazole and 1,4-bis[2-(5-phenyltriazolyl)]benzene. Another class of electron transport materials are oxadiazole metal chelates, such as bis[2-(2-hydroxyphenyl)-5-phenyl-1,3,4-oxadiazolato]zinc; bis[2-(2-hydroxyphenyl)-5-phenyl-1,3,4-oxadiazolato]beryllium; bis[2-(2-hydroxyphenyl)-5-(1-naphthyl)-1,3,4-oxadiazolato]zinc; bis[2-(2-hydroxyphenyl)-5-(-naphthyl)-1,3,4-oxadiazolato]beryllium; bis[5-biphenyl-2-(2-hydroxyphenyl)-1,3,4-oxadiazolato]zinc; bis[5-biphenyl-2-(2-hydroxyphenyl)-1,3,4-oxadiazolato]beryllium; bis(2-hydroxyphenyl)-5-phenyl-1,3,4-oxadiazolato]lithium; bis[2-(2-hydroxyphenyl)-5-p-tolyl-1,3,4-oxadiazolato]zinc; bis2-(2-hydroxyphenyl)-5-p-tolyl-1,3,4-oxadiazolato]beryllium; bis[5-(p-tert-butylphenyl)-2-(2-hydroxyphenyl)-1,3,4-oxadiazolato]zinc; bis[5-(p-tert-butylphenyl)-2-(2-hydroxyphenyl)-1,3,4-oxadiazolato]beryllium; bis[2-(2-hydroxyphenyl)-5-(3-fluorophenyl)-1,3,4-oxadiazolato]zinc; bis[2-(2-hydroxyphenyl)-5-(4-fluorophenyl)-1,3,4-oxadiazolato]zinc; bis[2-(2-hydroxyphenyl)-5-(4-fluorophenyl)-1,3,4-oxadiazolato]beryllium; bis[5-(4-chlorophenyl)-2-(2-hydroxyphenyl)-1,3,4-oxadiazolato]zinc; bis[2-(2-hydroxyphenyl)-5-(4-methoxyphenyl)-1,3,4-oxadiazolato]zinc; bis[2-(2-hydroxy-4-methylphenyl)-5-phenyl-1,3,4-oxadiazolato]zinc; bis[2-.alpha.-(2-hydroxynaphthyl)-5-phenyl-1,3,4-oxadiazolato]zinc; bis[2-(2-hydroxyphenyl)-5-p-pyridyl-1,3,4-oxadiazolato]zinc; bis[2-(2-hydroxyphenyl)-5-p-pyridyl-1,3,4-oxadiazolato]beryllium; bis[2-(2-hydroxyphenyl)-5-(2-thiophenyl)-1,3,4-oxadiazolato]zinc; bis[2-(2-hydroxyphenyl)-5-phenyl-1,3,4-thiadiazolato]zinc; bis[2-(2-hydroxyphenyl)-5-phenyl-1,3,4-thiadiazolato]beryllium; bis[2-(2-hydroxyphenyl)-5-(1-naphthyl)-1,3,4-thiadiazolato]zinc; and bis[2-(2-hydroxyphenyl)-5-(1-naphthyl)-1,3,4-thiadiazolato]beryllium, and the like.
In the organic EL device of the present invention,, the light-emitting layer may contain, in addition to the light-emitting organic material of the present invention, at least one of other light-emitting material, other dopant, other hole-injecting material and other electron-injecting material. For improving the organic EL device of the present invention in the stability against temperature, humidity and ambient atmosphere, a protective layer may be formed on the surface of the device, or the device as a whole may be sealed with a silicone oil, or the like. The electrically conductive material used for the anode of the organic EL device is suitably selected from those materials having a work function of greater than 4 eV. The electrically conductive material includes carbon, aluminum, vanadium, iron, cobalt, nickel, tungsten, silver, gold, platinum, palladium, alloys of these, metal oxides such as tin oxide and indium oxide used for ITO substrates or NESA substrates, and organic electroconducting polymers, such as polythiophene and polypyrrole.
The electrically conductive material used for the cathode is suitably selected from those having a work function of smaller than 4 eV. The electrically conductive material includes magnesium, calcium, tin, lead, titanium, yttrium, lithium, ruthenium, manganese, aluminum and alloys of these, while the electrically conductive material shall not be limited to these. Examples of the alloys include magnesium/silver, magnesium/indium and lithium/aluminum, while the alloys shall not be limited to these. Each of the anode and the cathode may have a layer structure formed of two layers or more as required.
For the effective light emission of the organic EL device, at least one of the electrodes is desirably sufficiently transparent in the light emission wavelength region of the device. Further, the substrate is desirably transparent as well. The transparent electrode is produced from the above electrically conductive material by a deposition method or a sputtering method such that a predetermined light transmittance is secured. The electrode on the light emission surface side has for instance a light transmittance of at least 10%. The substrate is not specially limited so long as it has adequate mechanical and thermal strength and has transparency. For example, it is selected from glass substrates and substrates of transparent resins such as a polyethylene substrate, a polyethylene terephthalate substrate, a polyether sulfone substrate and a polypropylene substrate.
In the organic EL device of the present invention, each layer can be formed by any one of dry film forming methods such as a vacuum deposition method, a sputtering method, a plasma method and an ion plating method and wet film forming methods such as a spin coating method, a dipping method and a flow coating method. The thickness of each layer is not specially limited, while each layer is required to have a proper thickness. When the layer thickness is too large, inefficiently, a high voltage is required to achieve predetermined emission of light. When the layer thickness is too small, the layer is liable to have a pinhole, etc., so that sufficient light emission brightness is hard to obtain when an electric field is applied. The thickness of each layer is for example in the range of from about 5 nm to about 10 μm, for instance about 10 nm to about 0.2 μm.
In the wet film forming method, a material for forming an intended layer is dissolved or dispersed in a proper solvent such as ethanol, chloroform, tetrahydrofuran and dioxane, and a thin film is formed from the solution or dispersion. The solvent shall not be limited to the above solvents. For improving the film formability and preventing the occurrence of pinholes in any layer, the above solution or dispersion for forming the layer may contain a proper resin and a proper additive. The resin that can be used includes insulating resins such as polystyrene, polycarbonate, polyarylate, polyester, polyamide, polyurethane, polysulfone, polymethyl methacrylate, polymethyl acrylate and cellulose, copolymers of these, photoconductive resins such as poly-N-vinylcarbozole and polysilane, and electroconducting polymers such as polythiophene and polypyrrole. The above additive includes an antioxidant, an ultraviolet absorbent and a plasticizer.
When the light-emitting organic material of the present invention is used in a light-emitting layer of an organic EL device, an organic EL device can be improved in organic EL device characteristics such as light emission efficiency and maximum light emission brightness. Further, the organic EL device of the present invention is remarkably stable against heat and electric current and gives a usable light emission brightness at a low actuation voltage. The problematic deterioration of conventional devices can be remarkably decreased.
The organic EL device of the present invention has significant industrial values since it can be adapted for a flat panel display of an on-wall television set, a flat light-emitting device, a light source for a copying machine or a printer, a light source for a liquid crystal display or counter, a display signboard and a signal light.

The material of the present invention can be used in the fields of an organic EL device, an electrophotographic photoreceptor, a photoelectric converter, a solar cell, an image sensor, dye lasers and the like.

The triazine compounds of formula I are novel. Hence, a further subject of the present invention is directed to triazine compounds of formula I,

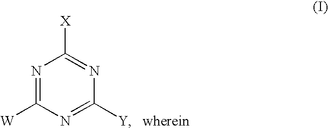

W is a group of formula

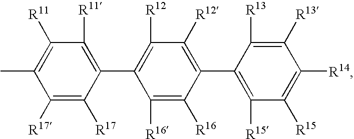

X and Y are independently of each other an aryl group or a heteroaryl group, especially a group of formula

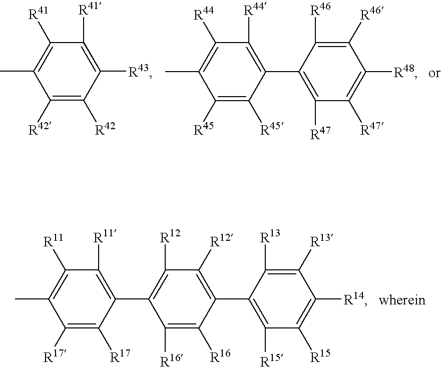

R¹¹, R^{11′}, R¹², R^{12′}, R¹³, R^{13′}, R¹⁵, R^{15′}, R¹⁶, R^{16′}, R¹⁷, R^{17′}, R⁴¹, R^{41′}, R⁴², R^{42′}, R⁴⁴, R^{44′}, R⁴⁵, R^{45′}, R⁴⁶, R^{46′}, R⁴⁷ and R^{47′} are independently of each other H, E, C₆-C₁₈aryl; C₆-C₁₈aryl which is substituted by G; C₁-C₁₈alkyl; C₁-C₁₈alkyl which is substituted by E and/or interrupted by D; C₇-C₁₈aralkyl; or C₇-C₁₈aralkyl which is substituted by G; or
R^{11′} and R¹², R^{12′} and R¹³, R^{15′} and R¹⁶, R^{16′} and R¹⁷, R^{44′} and R⁴⁶ and/or R^{45 ′} and R⁴⁷ are each a divalent group L¹ selected from an oxygen atom, an sulfur atom, >CR¹⁸R¹⁹>SiR¹⁸R¹⁹, or

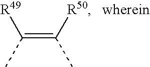

R¹⁸ and R¹⁹ are independently of each other C₁-C₁₈alkyl; C₁-C₁₈alkoxy, C₆-C₁₈aryl; C₇-C₁₈aralkyl; R¹¹ and R^{11′}, R¹² and R^{12′}, R¹³ and R^{13′} and R¹⁴, R^{14′} and R¹⁵, R¹⁵ and R^{15′}, R¹⁶ and R^{16′}, R^{17′} and R¹⁷, R⁴¹ and R^{41′}, R⁴² and R^{42′}, R^{42′} and R⁴³, R^{41′} and R⁴³, R⁴⁴ and R^{44′}, R⁴⁵ and R^{45′}, R⁴⁶ and R^{46′}, R⁴⁷ and R^{47′}, R^{46′}and R⁴⁸ and/or R^{47′} and R⁴⁸ are each a divalent group

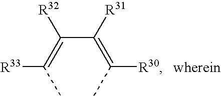

R³⁰, R³¹, R³², R³³, R⁴⁹ and R⁵⁰ are independently of each other H, C₁-C₁₈alkyl; C₁-C₁₈alkyl, which is substituted by E and/or interrupted by D; E; C₆-C₁₈aryl; C₆-C₁₈aryl, which is substituted by E;
R¹⁴ is H, C₂-C₃₀heteroaryl, C₂-C₃₀heteroaryl, which is substituted by G, C₆-C₃₀aryl, or C₆-C₃₀aryl, which is substituted by G, C₁-C₁₈alkyl; or C₁-C₁₈alkyl which is substituted by E and/or interrupted by D; especially

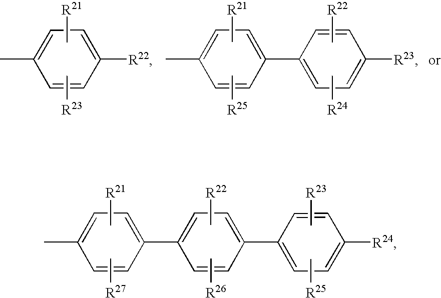

wherein R²¹, R²², R²³, R²⁴, R²⁵, R²⁶ and R²⁷ are independently of each other H, E, C₁-C₁₈alkyl; C₁-C₁₈alkyl which is substituted by E and/or interrupted by D; E; C₇-C₁₈aralkyl; C₇-C₁₈aralkyl which is substituted by G;
R⁴³ and R⁴⁸ are independently of each other H, E; C₁-C₁₈alkyl; C₁-C₁₈alkyl, which is substituted by E and/or interrupted by D; C₂-C₃₀heteroaryl; C₂-C₃₀heteroaryl, which is substituted by G, C₇-C₁₈aralkyl; or C₇-C₁₈aralkyl which is substituted by G;
D is —CO—; —COO—; —OCOO—; —S—; —SO—; —SO₂—; —O—; —NR⁵—; SiR⁶¹R⁶²—; —POR⁵—; —CR⁶³═CR⁶⁴—; or —C═C—;
E is —OR⁵; —SR⁵; —NR⁵R⁶; —COR⁸; —COOR⁷; —OCOOR⁷, —CONR⁵R⁶; —CN; or halogen;
G is E, or C₁-C₁₈alkyl,
wherein R⁵ and R⁶ are independently of each other C₆-C₁₈aryl; C₆-C₁₈aryl which is substituted by C₁-C₁₈alkyl, C₁-C₁₈alkoxy; or C₁-C₁₈alkyl which is interrupted by —O—; or
R⁵ and R⁶ together form a five or six membered ring, in particular

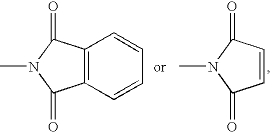

R⁷ is C₆-C₁₈aryl; C₆-C₁₈aryl which is substituted by C₁-C₁₈alkyl, C₁-C₁₈alkoxy; or C₁-C₁₈alkyl which is interrupted by —O—;
R⁸ is C₇-C₁₂alkylaryl; C₁-C₁₈alkyl; or C₁-C₁₈alkyl which is interrupted by —O—;
R⁶¹ and R⁶² are independently of each other C₆-C₁₈aryl; C₆-C₁₈aryl which is substituted by C₁-C₁₈alkyl, C₁-C₁₈alkoxy; or C₁-C₁₈alkyl which is interrupted by —O—, and
R⁶³ and R⁶⁴ are independently of each other H, C₆-C₁₈aryl; C₆-C₁₈aryl which is substituted by C₁-C₁₈alkyl, C₁-C₁₈alkoxy; or C₁-C₁₈alkyl which is interrupted by —O—.

W is preferably a group of formula

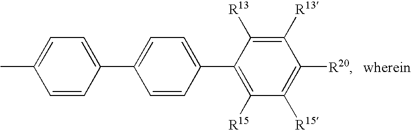

R¹³, R^{13′}, R¹⁵ and R^{15′} are H and R²⁰ is H, especially

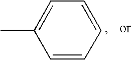

R¹³ and R¹⁵ are H, R^{13′} and R¹⁵ are independently of each other H, C₁-C₈alkyl, or C₁-C₈alkoxy, and R²⁰ is H, C₁-C₈alkyl, or C₁-C₈alkoxy; or

### R¹³, R¹⁵ and R^{15′} are H, and R ¹³ and R²⁰ are

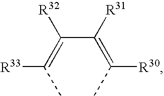

### R²⁰, R¹⁵ and R^{15′} are H, and R¹³ and R^{13′} are

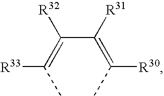

wherein
R³⁰, R³¹, R³² and R³³ are H, C₁-C₈alkyl, or C₁-C₈alkoxy, and
X and Y are as defined above.

According to the present invention at least W, preferably W and Y, most preferred W, Y and X are a group of formula

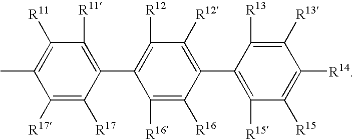

Accordingly, in one preferred embodiment of the present invention the triazine compound is a compound of formula I, wherein W and Y are independently of each other a group of formula

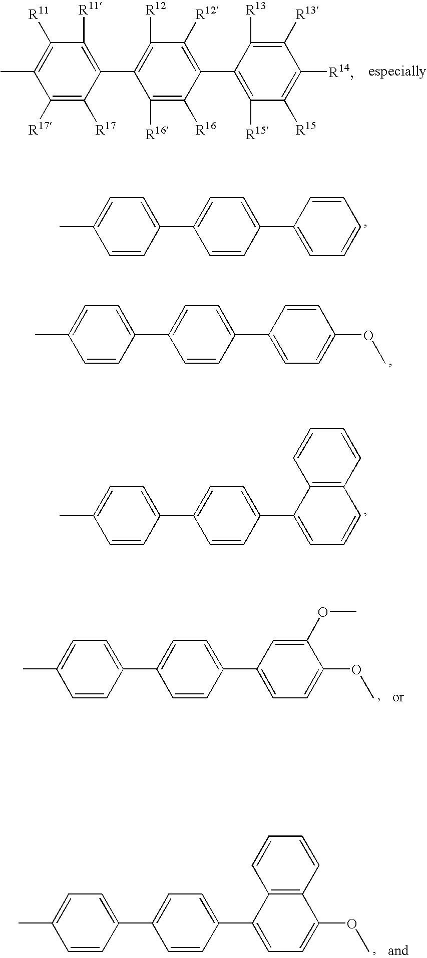

X is a group of formula

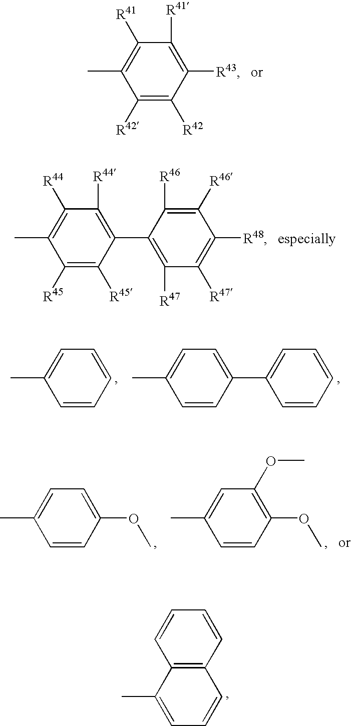

wherein R¹¹, R^{11′}, R¹², R^{12′}, R¹³, R^{13′}, ¹⁴, R¹⁵, R^{15′}, R¹⁶, R^{16′},R¹⁷, R^{17′}, R⁴¹, R^{41′}, R⁴², R^{42′}, R⁴⁴ R^{44′}, R⁴⁵, R^{45′}, R⁴⁶, R^{46′}, R⁴⁷, R^{47′}, R⁴³ and R⁴⁸ are as defined above, and are especially H, C₁-C₈alkyl, C₁-C₈alkoxy, or phenyl.

R¹¹, R^{11′},R¹², R^{12′}, R¹³, R^{13′}, R¹⁵, R^{15′}, R¹⁶, R^{16′}, R¹⁷ and R^{17′}, R⁴¹, R^{41′}, R⁴², R^{42′}, R⁴⁴, R^{44′}, R⁴⁵, R^{45′}, R⁴⁶, R^{46′}, R⁴⁷, and R^{47′} as well as R¹⁴, R⁴³, and R⁴⁸ are preferably independently of each other H, E; or C₁-C₈alkyl, especially H, C₁-C₄alkyl, C₁-C₄alkoxy, or phenyl; wherein E is —OR⁵; —SR⁵; —NR⁵R⁶; —COR⁸; —COOR⁷; —CONR⁵R⁶; —CN; —OCOOR⁷; or halogen; wherein R⁵ and R⁶ are independently of each other C₆-C₁₂ary, or C₁-C₈alkyl;

R⁷ is C₇-C₁₂alkylaryl, or C₁-C₈alkyl; and
R⁸ is C₆-C₁₂aryl; or C₁-C₈alkyl, or

### R¹¹ and R^{11′}, R¹² and R^{12′}, R¹³ and R^{13′}, R^{13′} and R¹⁴, R^{14′} and R^{41′}, R^{41′} and R^{44′},

R⁴⁶ and R^{46′}, R^{46′} and R⁴⁸ and/or R^{47′} and R⁴⁸ are each a divalent group

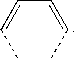

In one more preferred embodiment of the present invention W, X and Y are independently of each other a group of formula

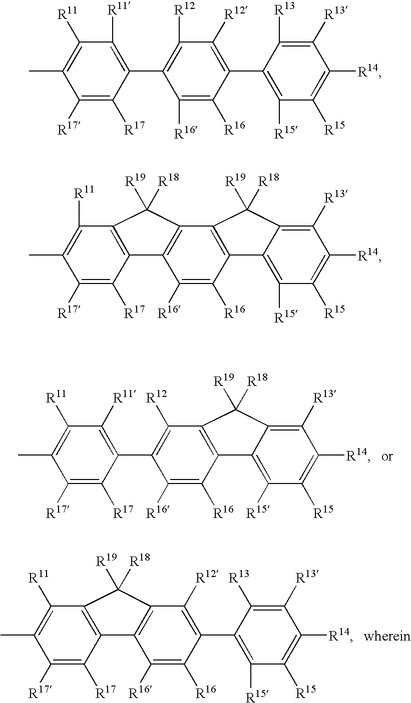

R¹¹, R^{11′}, R¹², R^{12′}, R¹³, R^{13′}, R¹⁵, R^{15′}, R¹⁶, R^{16′}, R¹⁷ and R^{17′}are independently of each other H, C₆-C₁₈aryl; C₆-C₁₈aryl which is substituted by G; E, C₁-C₁₈alkyl; C₁-C₁₈alkyl which is substituted by E and/or interrupted by D; C₇-C₁₈aralkyl; C₇-C₁₈aralkyl which is substituted by G; and D, E, G, R¹⁴, R¹⁸ and R¹⁹ are as defined above, or

W is a group of the formula —W¹—W²—W³,
X is a group of the formula —X¹—X²—X³ and
Y is a group of the formula —Y¹—Y²—Y³, wherein W¹, W², X¹, X², Y¹ and Y² are independently of each other a group of formula

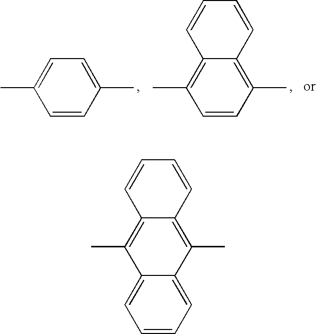

and W³, X³ and Y³ are independently of each other a group of formula

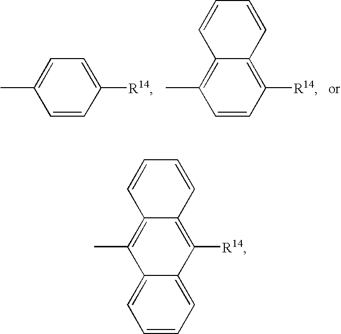

wherein R¹⁴ is as defined above.

W, X and Y can be different, but have preferably the same meaning.

Triazine compounds of formula I are preferred, wherein R¹¹, R^{11′}, R¹², R^{12′}, R¹³, R^{13′}, R¹⁵, R^{15′}, R¹⁶, R^{16′}, R¹⁷ and R^{17′}, R⁴¹, R41′, R⁴², R^{42′}, R⁴⁴, R^{44′}, R⁴⁵, R^{45′}, R⁴⁶, R^{46′}, R⁴⁷, and R^{47′} are independently of each other H, E; or C₁-C₈alkyl; wherein

E is —OR⁵; —SR⁵; —NR⁵R⁶; —COR⁸; —COOR⁷; —CONR⁵R⁶; —CN; —OCOOR⁷; or halogen; wherein R⁵ and R⁶ are independently of each other C₆-C₁₂aryl; or C₁-C₈alkyl;
R⁷ is C₇-C₁₂ alkylaryl, or C₁-C₈alkyl; and
R⁸ is C₆-C₁₂aryl, or C₁-C₈alkyl.

Especially preferred are triazine compounds of formula I, wherein

W, X and Y are independently of each other a group of formula

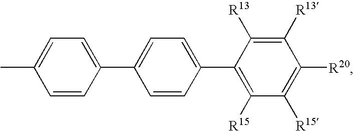

wherein R¹³, R^{13′}, R¹⁵ and R^{15′} are H and R²⁰ is H, especially

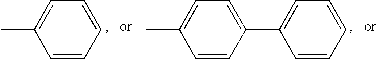

R¹³ and R¹⁵ are H, R^{13′} and R^{15′} are independently of each other H, C₁-C₈alkyl, or C₁-C₈alkoxy, and R²⁰ is H, C₁-C₈alkyl, or C₁-C₈alkoxy; or

### R¹³, R¹⁵ and R^{15′} are H, and R^{13′} and R²⁰ are

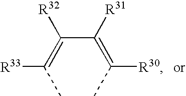

### R²⁰, R¹⁵ and R^{15′} are H, and R¹³ and R^{13′} are

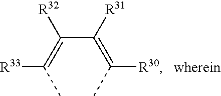

R³⁰, R³¹, R³² and R³³ are H, C₁-C₈alkyl, or C₁-C₈alkoxy; or wherein
W, X and Y are independently of each other a group of formula

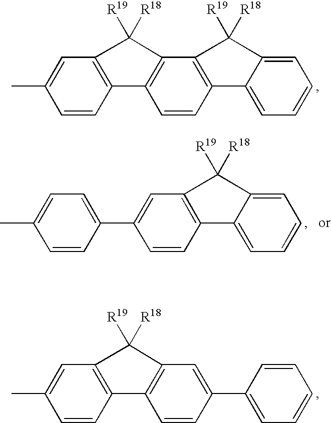

wherein R¹⁸ and R¹⁹ are independently of each other C₁-C₈alkyl.

Specific examples of preferred triazine compounds are:

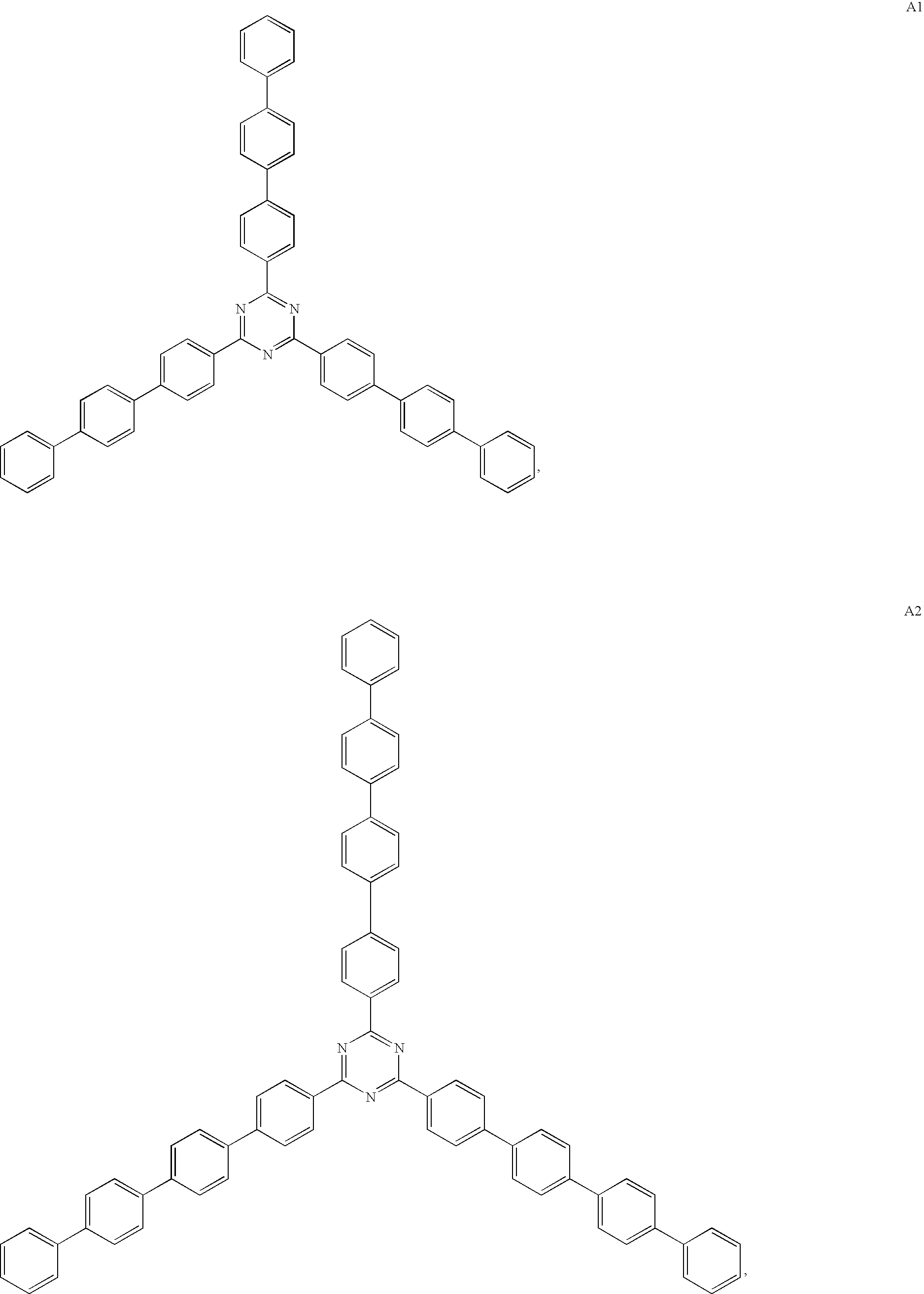

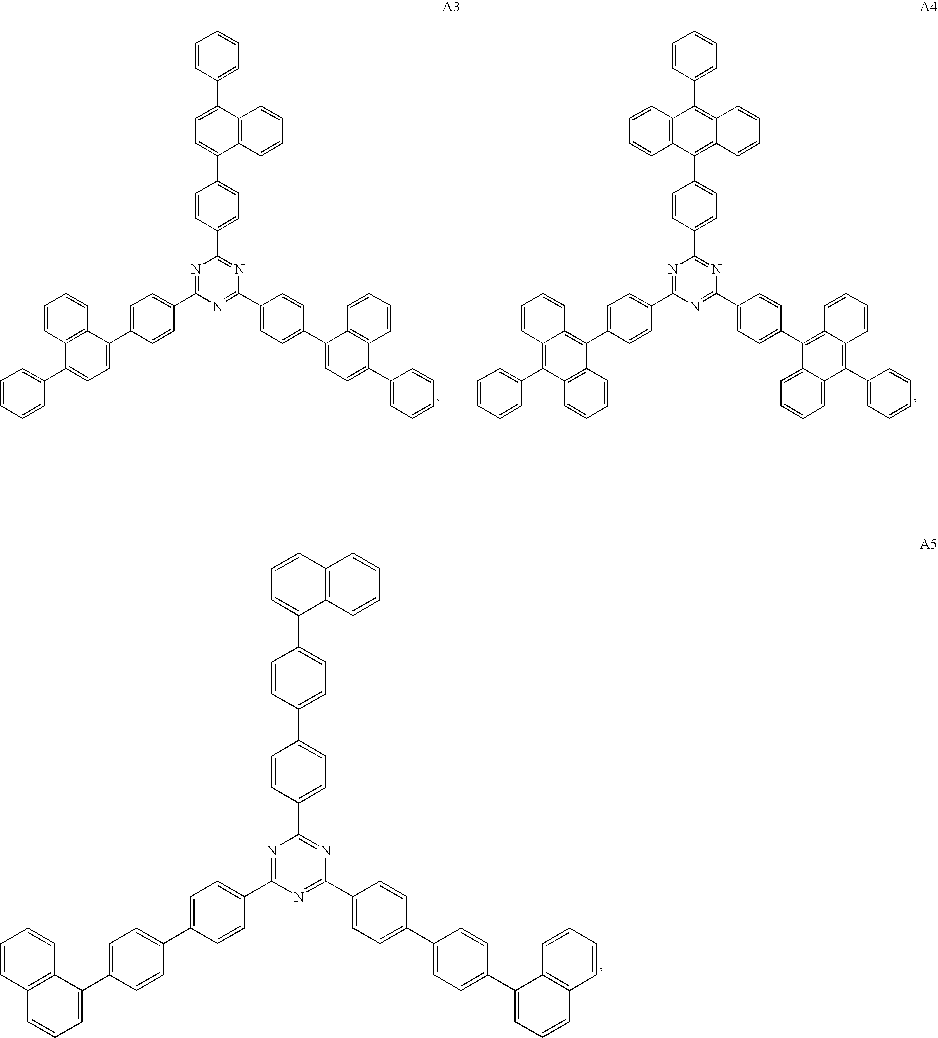

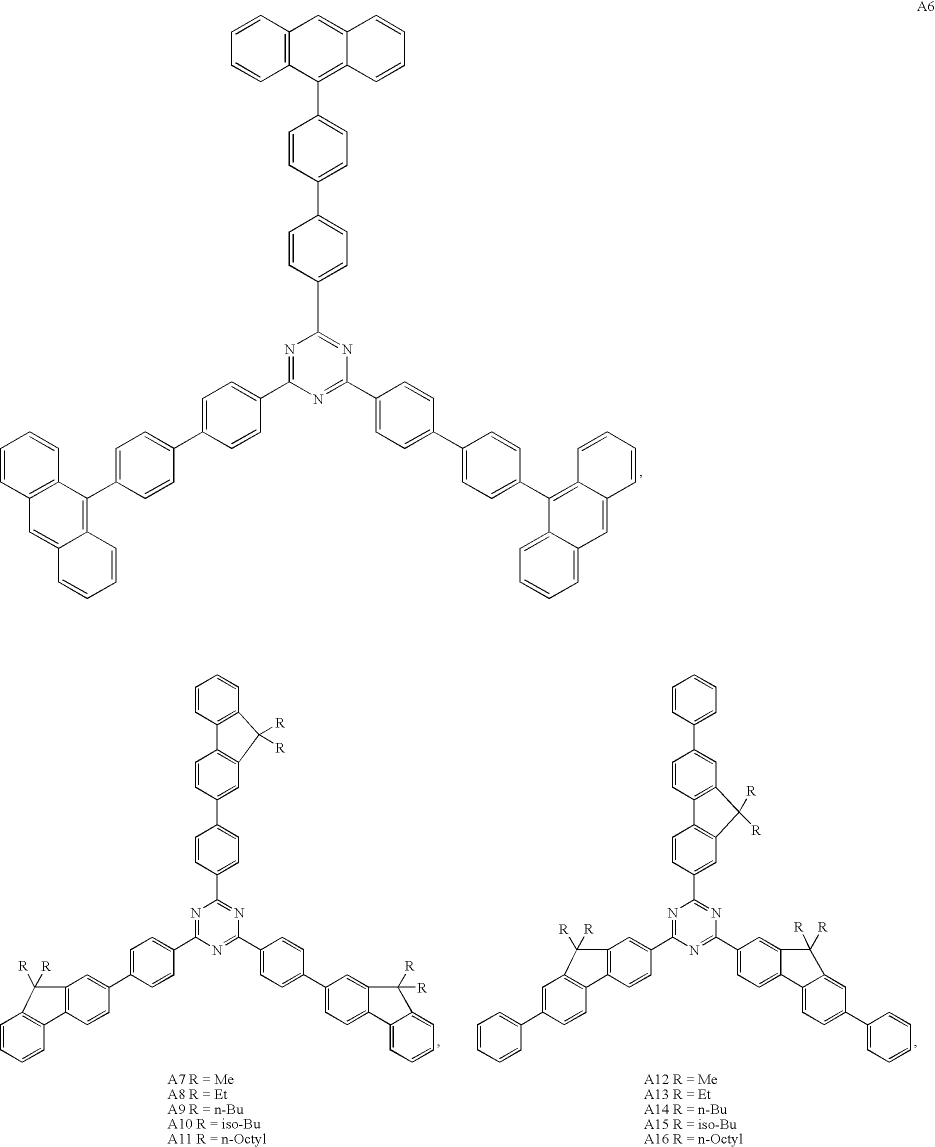

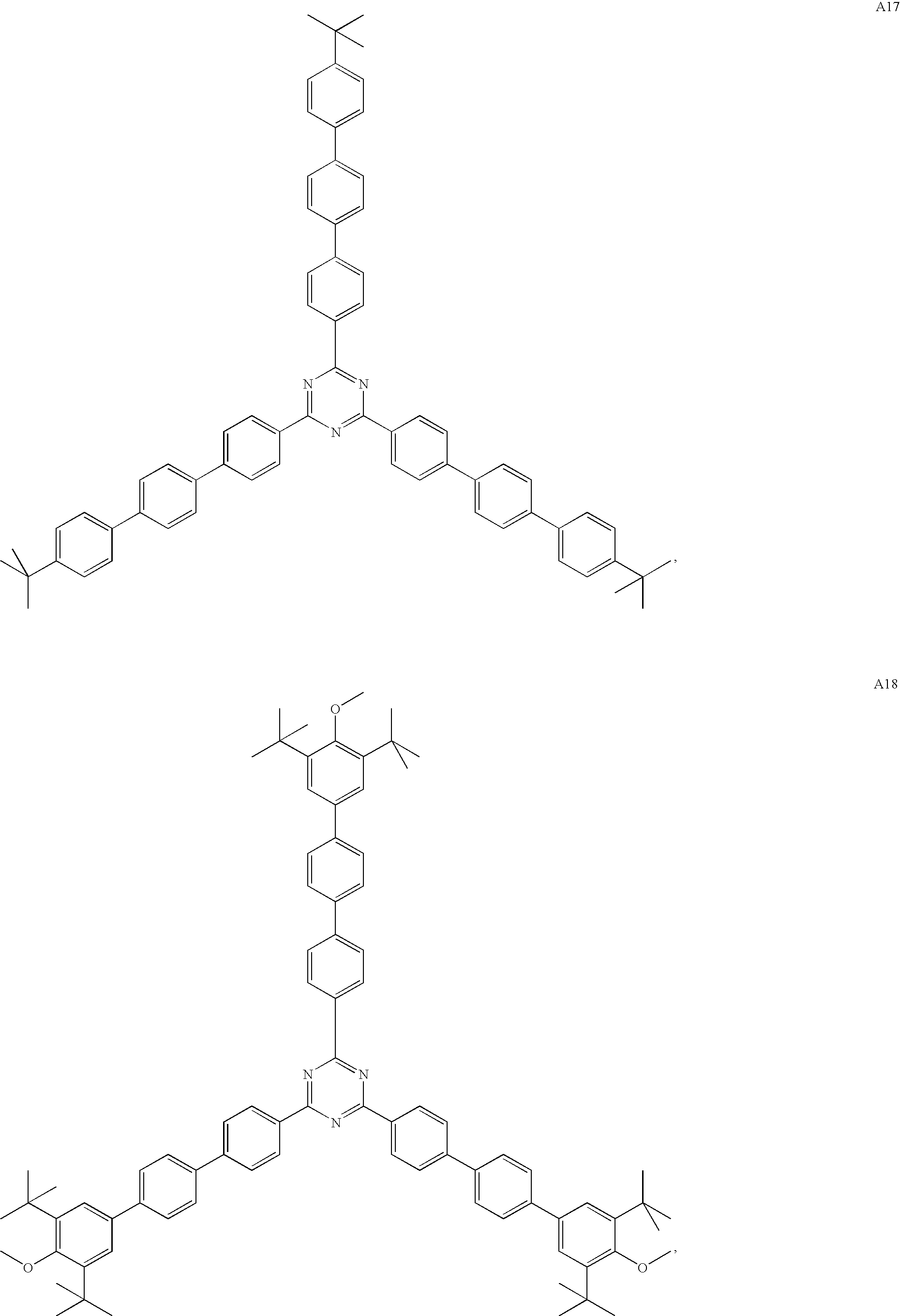

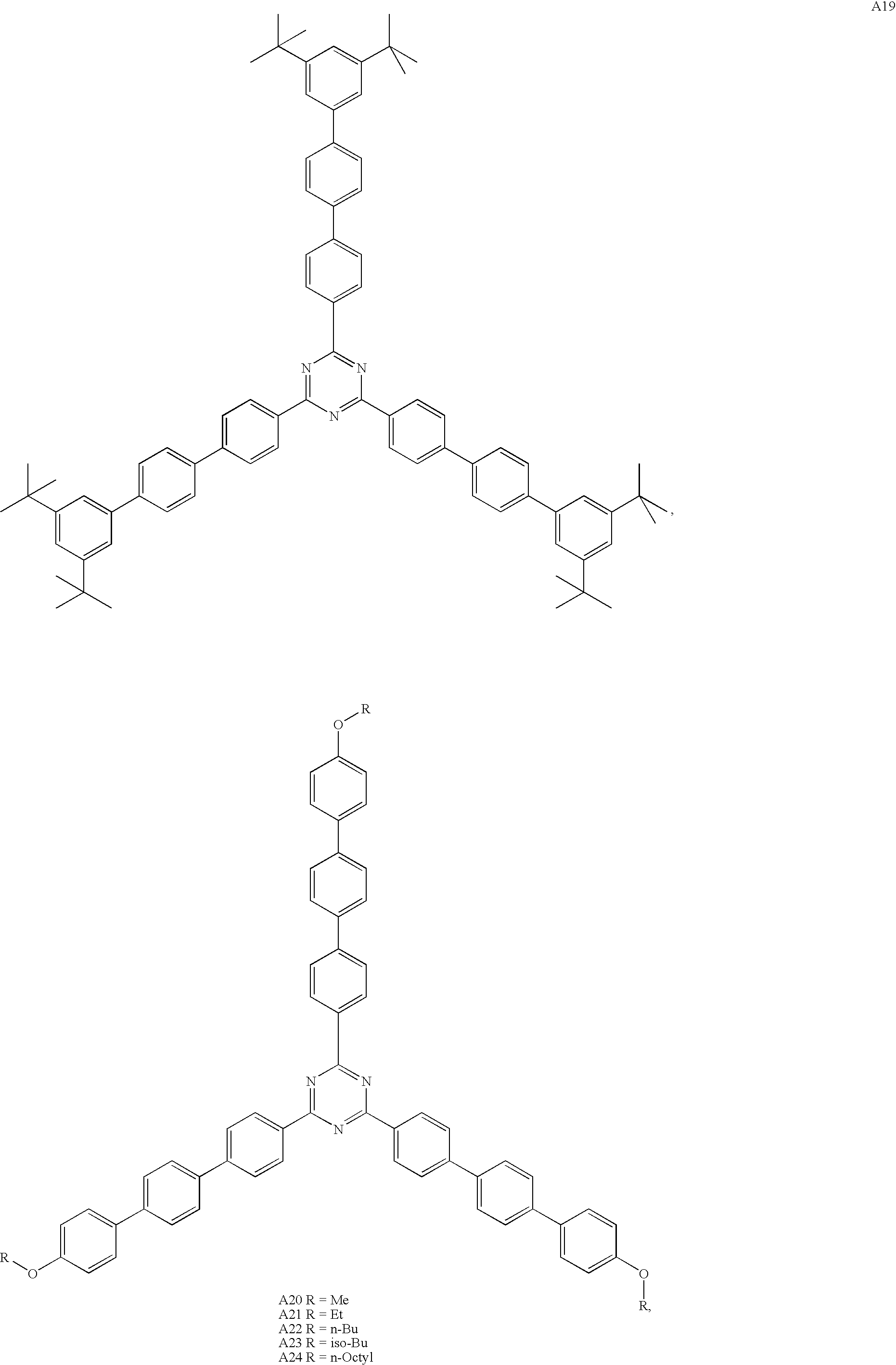

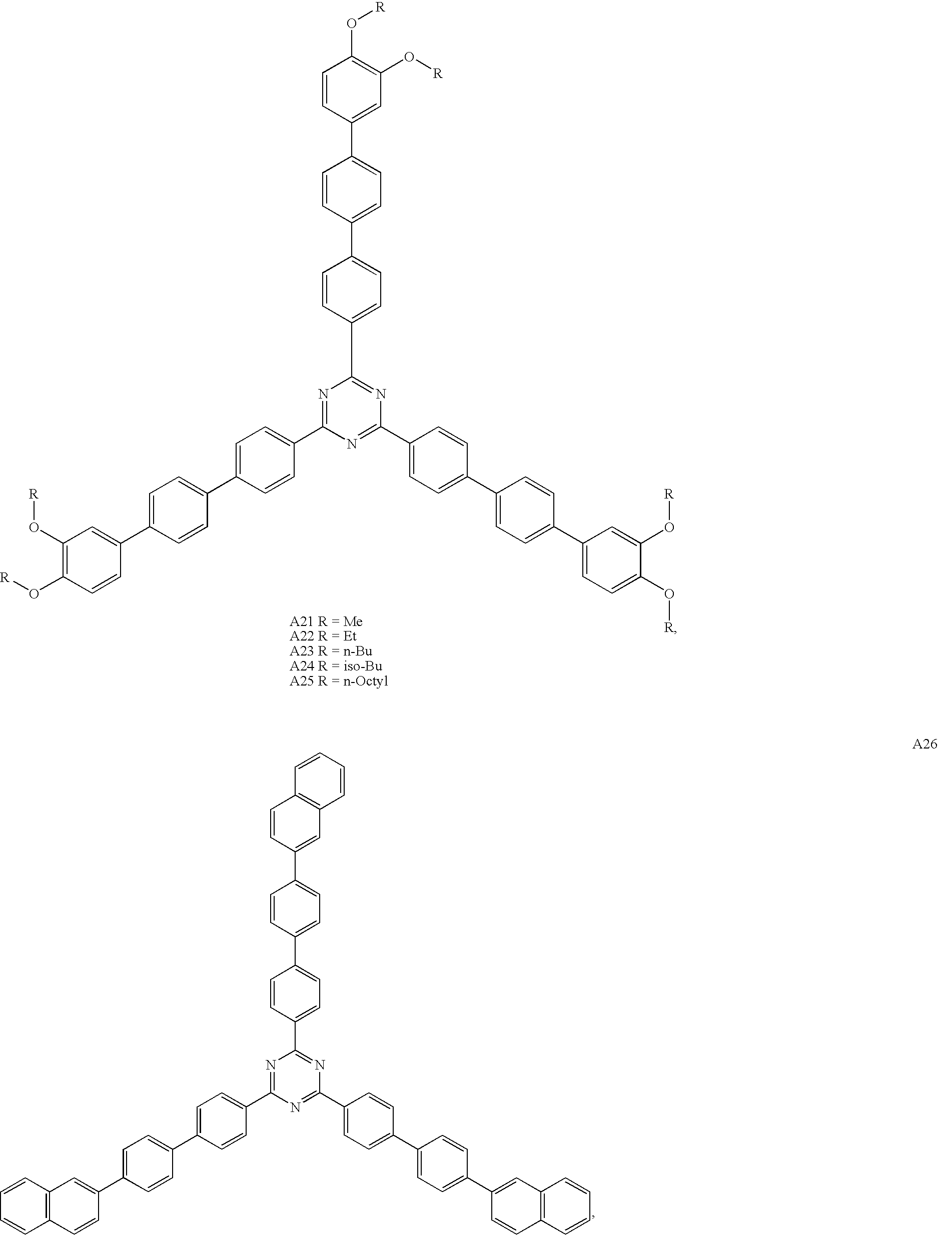

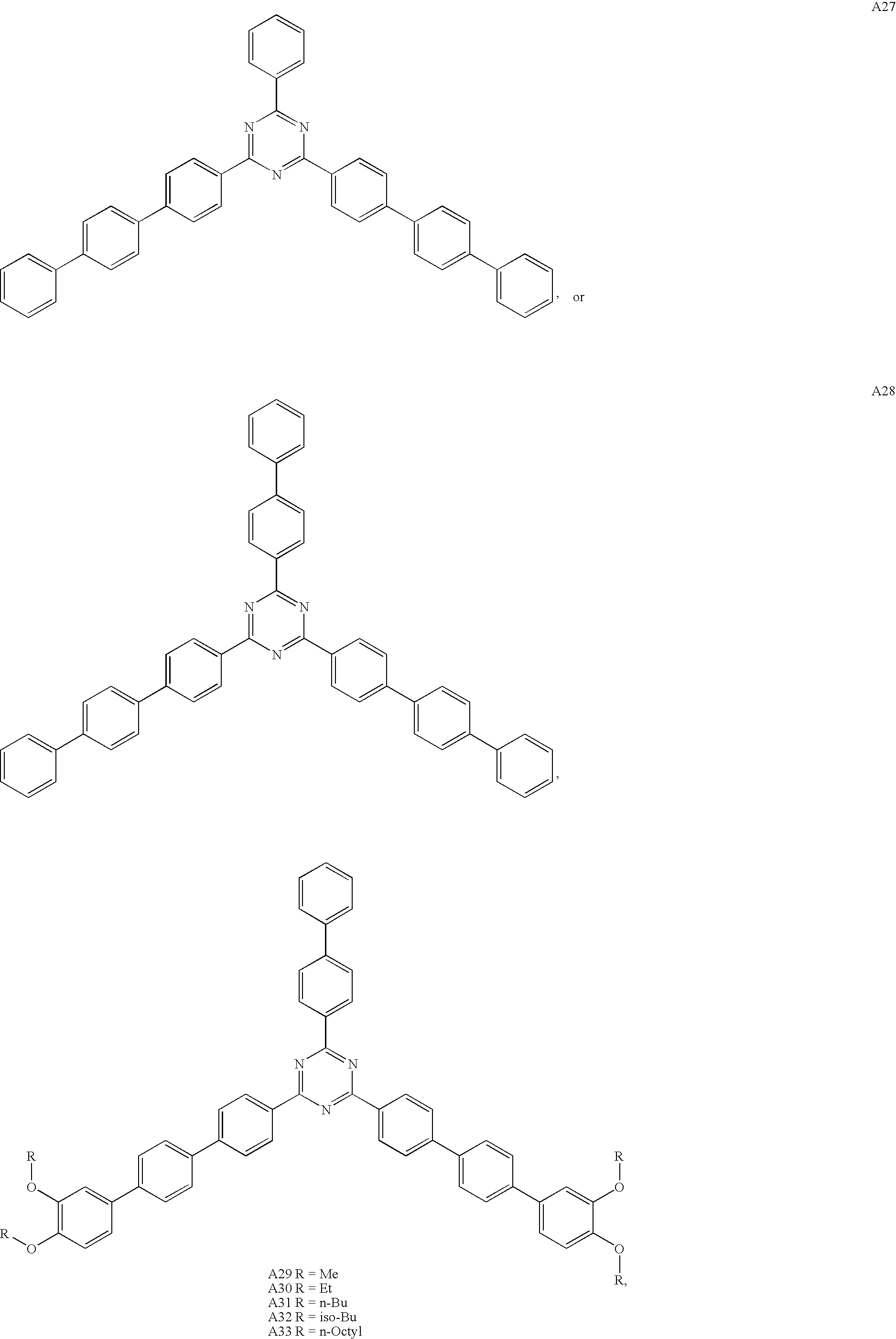

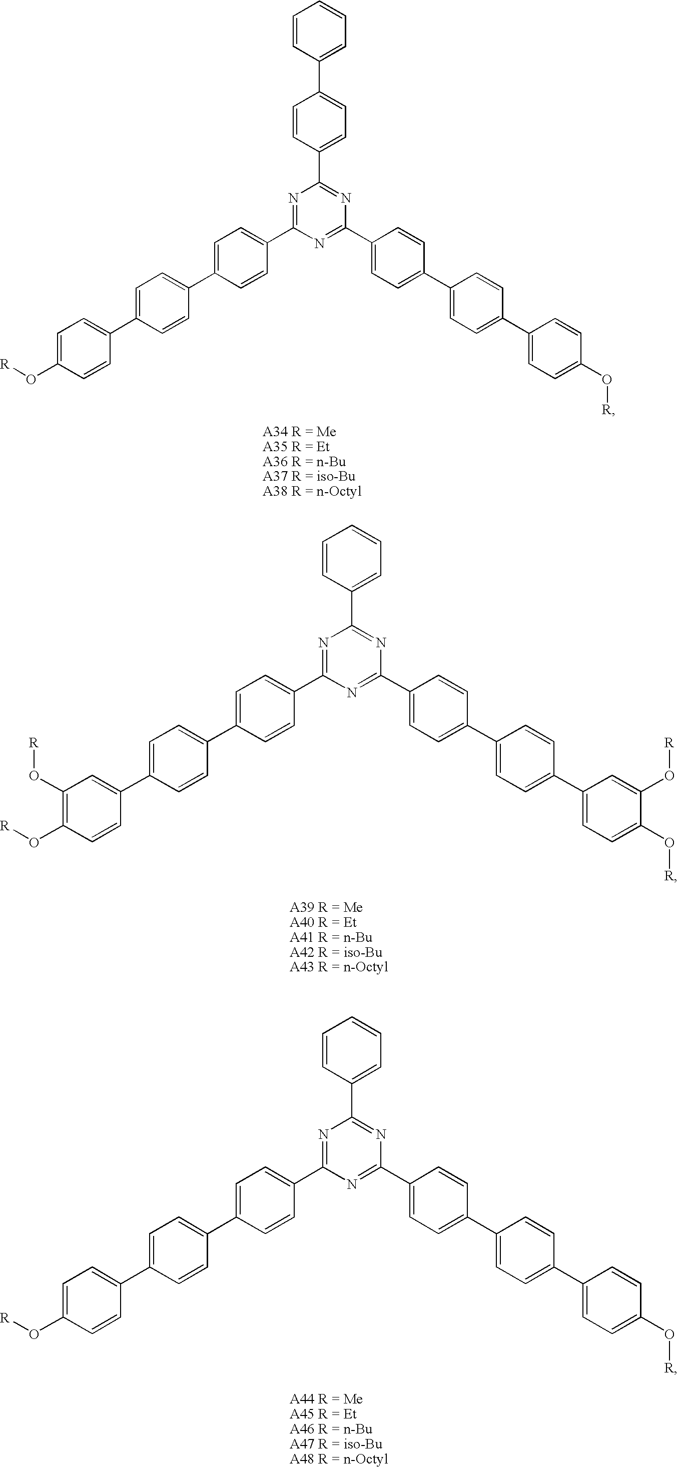

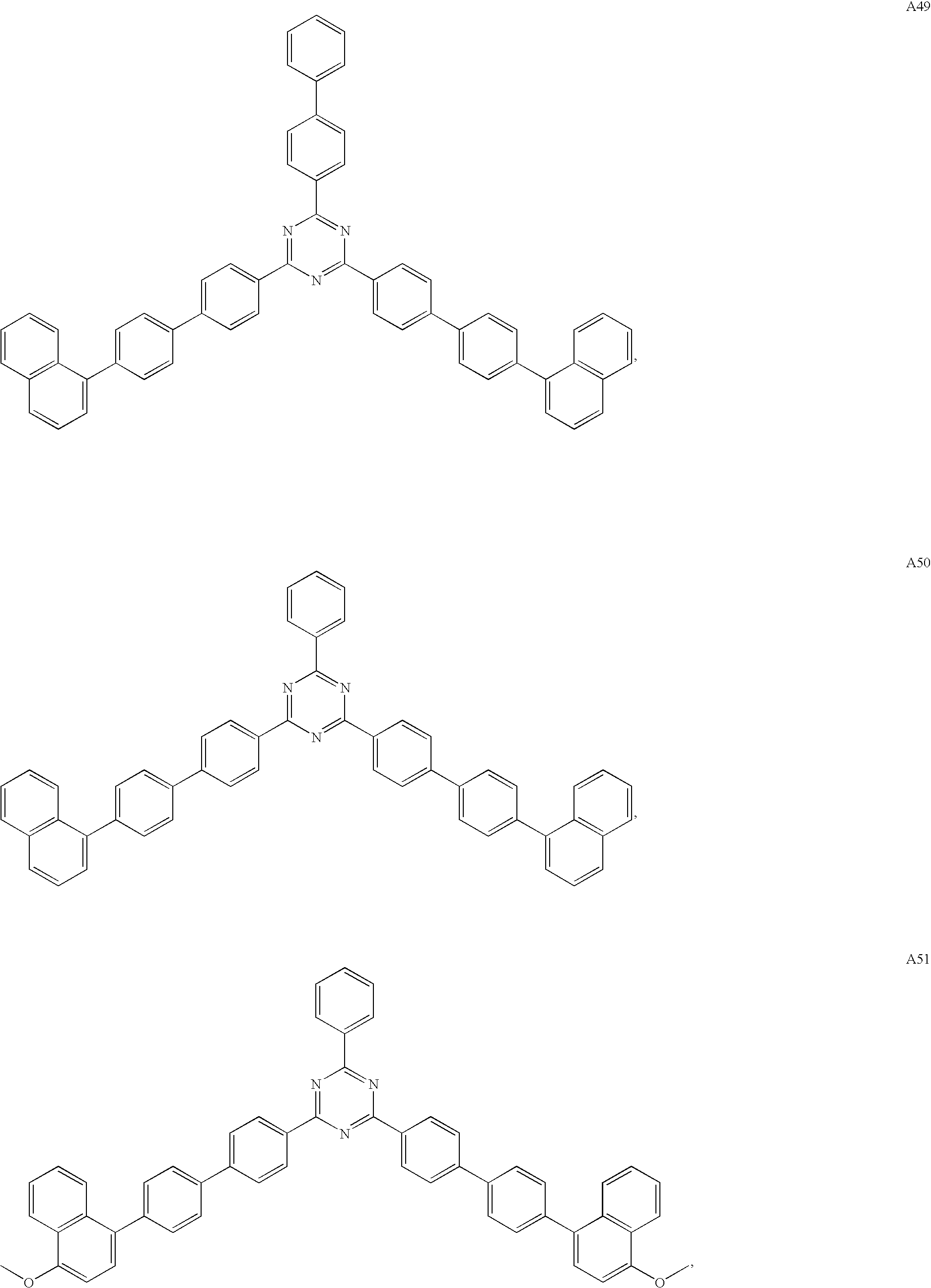

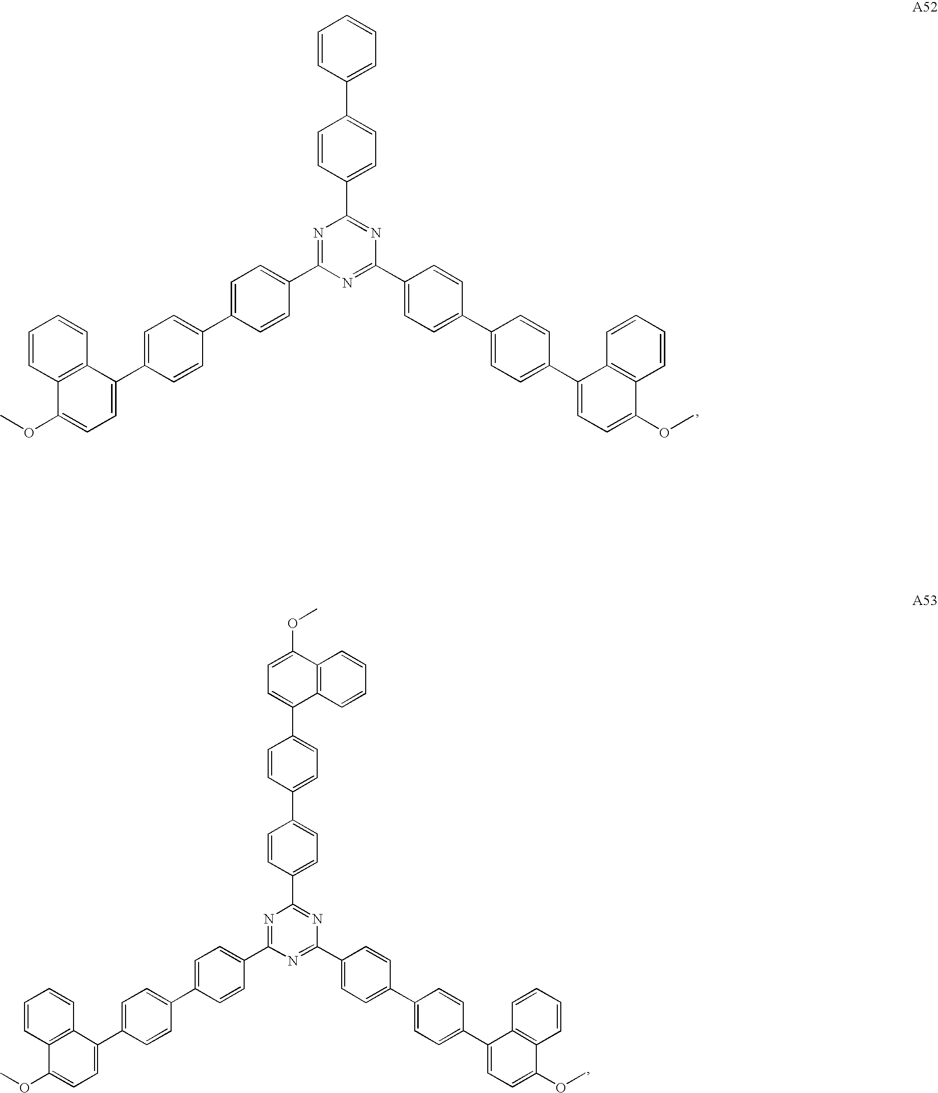

The present triazine compounds show a high solid state fluorescence in the desired wavelength range and can be prepared according to or analogous to known procedures. The triazine compounds of the present invention of the formula:

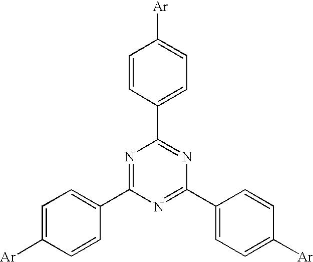

can, for example, be prepared according to a process, which comprises reacting a derivative of formula

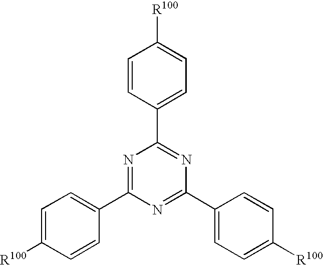

wherein R¹⁰⁰ stands for halogen such as chloro or bromo, preferably bromo, or E having the meaning of

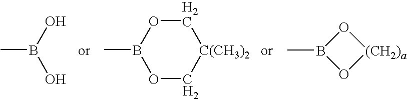

wherein a is 2 or 3,
with boronic acid derivative

### E-Ar,

or—in case R¹⁰⁰ is not halogen—

### Hal-Ar,

wherein Hal stands for halogen, preferably for bromo,
wherein Ar is C₁₂-C₃₀-aryl, which can be substituted, especially

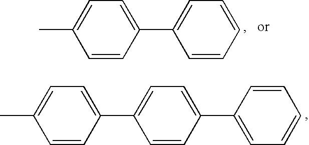

in the presence of an allylpalladium catalyst of the μ-halo(triisopropylphosphine)(η³-allyl)palladium(II) type (see for example W099/47474).

Accordingly, unsymmetrical substituted triazine compounds of the present invention of the formula:

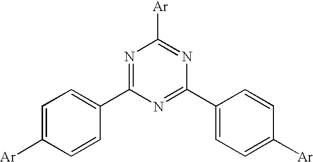

can, for example, be prepared according to a process, which comprises reacting a derivative of formula

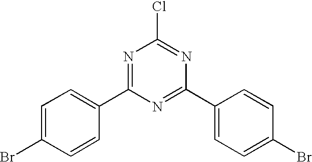

with a boronic acid derivative E-Ar, wherein E is as defined above.

C₁-C₁₈alkyl is a branched or unbranched radical such as for example methyl, ethyl, propyl, isopropyl, n-butyl, sec-butyl, isobutyl, tert-butyl, 2-ethylbutyl, n-pentyl, isopentyl, 1-methylpentyl, 1,3-dimethylbutyl, n-hexyl, 1-methylhexyl, n-heptyl, isoheptyl, 1,1,3,3-tetramethylbutyl, 1-methylheptyl, 3-methylheptyl, n-octyl, 2-ethylhexyl, 1,1,3-trimethylhexyl, 1,1,3,3-tetramethylpentyl, nonyl, decyl, undecyl, 1-methylundecyl, dodecyl, 1,1,3,3,5,5-hexamethylhexyl, tridecyl, tetradecyl, pentadecyl, hexadecyl, heptadecyl, or octadecyl.

C₁-C₁₈Alkoxy radicals are straight-chain or branched alkoxy radicals, e.g. methoxy, ethoxy, n-propoxy, isopropoxy, n-butoxy, sec-butoxy, tert-butoxy, amyloxy, isoamyloxy or tert-amyloxy, heptyloxy, octyloxy, isooctyloxy, nonyloxy, decyloxy, undecyloxy, dodecyloxy, tetradecyloxy, pentadecyloxy, hexadecyloxy, heptadecyloxy and octadecyloxy.

C₂-C₁₈Alkenyl radicals are straight-chain or branched alkenyl radicals, such as e.g. vinyl, allyl, methallyl, isopropenyl, 2-butenyl, 3-butenyl, isobutenyl, n-penta-2,4-dienyl, 3-methyl-but-2-enyl, n-oct-2-enyl, n-dodec-2-enyl, isododecenyl, n-dodec-2-enyl or n-octadec-4-enyl.

C₂₋₂₄Alkynyl is straight-chain or branched and preferably C₂₋₈alkynyl, which may be unsubstituted or substituted, such as, for example, ethynyl, 1-propyn-3-yl, 1-butyn-4-yl, 1-pentyn-5-yl, 2-methyl-3-butyn-2-yl, 1,4-pentadiyn-3-yl, 1,3-pentadiyn-5-yl, 1-hexyn-6-yl, cis-3-methyl-2-penten-4-yn-1-yl, trans-3-methyl-2-penten-4-yn-1-yl, 1,3-hexadiyn-5-yl, 1-octyn-8-yl, 1-nonyn-9-yl, 1-decyn-10-yl or 1-tetracosyn-24-yl,

C₄-C₁₈cycloalkyl is preferably C₅-C₁₂cycloalkyl, such as, for example, cyclopentyl, cyclohexyl, cycloheptyl, cyclooctyl, cyclononyl, cyclodecyl, cyclododecyl. Cyclohexyl and cyclododecyl are most preferred.

The term “aryl group” is typically C₆-C₃₀aryl, such as phenyl, indenyl, azulenyl, naphthyl, biphenyl, terphenylyl or quadphenylyl, as-indacenyl, s-indacenyl, acenaphthylenyl, phenanthryl, fluoranthenyl, triphenlenyl, chrysenyl, naphthacen, picenyl, perylenyl, pentaphenyl, hexacenyl, pyrenyl, or anthracenyl, preferably phenyl, 1-naphthyl, 2-naphthyl, 9-phenanthryl, 2- or 9-fluorenyl, 3- or 4-biphenyl, which may be unsubstituted or substituted. Examples of C₆-C₁₈aryl are phenyl, 1-naphthyl, 2-naphthyl, 3- or 4-biphenyl, 9-phenanthryl, 2- or 9-fluorenyl, which may be unsubstituted or substituted.

C₇-C₂₄aralkyl radicals are preferably C₇-C₁₈aralkyl radicals, which may be substituted, such as, for example, benzyl, 2-benzyl-2-propyl, β-phenyl-ethyl, α,α-dimethylbenzyl, ω-phenyl-butyl, ω,ω-dimethyl-ω-phenyl-butyl, ω-phenyl-dodecyl, ω-phenyl-octadecyl, ω-phenyl-eicosyl or ω-phenyl-docosyl, preferably C₇-C₁₈aralkyl such as benzyl, 2-benzyl-2-propyl, β-phenyl-ethyl, α,α-dimethylbenzyl, ω-phenyl-butyl, ω,ω-dimethyl-ω-phenyl-butyl, ω-phenyl-dodecyl or ω-phenyl-octadecyl, and particularly preferred C₇-C₁₂aralkyl such as benzyl, 2-benzyl-2-propyl, β-phenyl-ethyl, α,α-dimethylbenzyl, ω-phenyl-butyl, or ω,ω-dimethyl-ω-phenyl-butyl, in which both the aliphatic hydrocarbon group and aromatic hydrocarbon group may be unsubstituted or substituted.

C₇-C₁₂alkylaryl is, for example, a phenyl group substituted with one, two or three C₁-C₆alkyl groups, such as, for example, 2-, 3-, or 4-methylphenyl, 2-, 3-, or 4-ethylphenyl, 3-, or 4-isopropylphenyl, 3,4-dimethylphenyl, 3,5-dimethylphenyl, or 3,4,5-trimethylphenyl.

The term “heteroaryl group”, especially C₂-C₃₀heteroaryl, is a ring, wherein nitrogen, oxygen or sulfur are the possible hetero atoms, and is typically an unsaturated heterocyclic radical with five to 18 atoms having at least six conjugated n-electrons such as thienyl, benzo[b]thienyl, dibenzo[b,d]thienyl, thianthrenyl, furyl, furfuryl, 2H-pyranyl, benzofuranyl, isobenzofuranyl, 2H-chromenyl, xanthenyl, dibenzofuranyl, phenoxythienyl, pyrrolyl, imidazolyl, pyrazolyl, pyridyl, bipyridyl, triazinyl, pyrimidinyl, pyrazinyl, 1H-pyrrolizinyl, isoindolyl, pyridazinyl, indolizinyl, isoindolyl, indolyl, 3H-indolyl, phthalazinyl, naphthyridinyl, quinoxalinyl, quinazolinyl, cinnolinyl, indazolyl, purinyl, quinolizinyl, chinolyl, isochinolyl, phthalazinyl, naphthyridinyl, chinoxalinyl, chinazolinyl, cinnolinyl, pteridinyl, carbazolyl, 4aH-carbazolyl, carbolinyl, benzotriazolyl, benzoxazolyl, phenanthridinyl, acridinyl, perimidinyl, phenanthrolinyl, phenazinyl, isothiazolyl, phenothiazinyl, isoxazolyl, furazanyl or phenoxazinyl, preferably the above-mentioned mono- or bicyclic heterocyclic radicals, which may be unsubstituted or substituted.

Halogen is fluorine, chlorine, bromine and iodine.

Examples of a five or six membered ring formed by R⁵ and R⁶ are heterocycloalkanes or heterocycloalkenes having from 3 to 5 carbon atoms which can have one additional hetero atom selected from nitrogen, oxygen and sulfur, for example

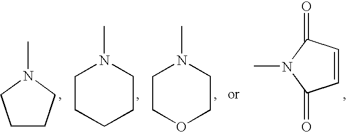

which can be part of a bicyclic system, for example

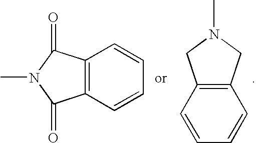

Possible substituents of the above-mentioned groups are C₁-C₈alkyl, a hydroxyl group, a mercapto group, C₁-C₈alkoxy, C₁-C₈alkylthio, halogen, halo-C₁-C₈alkyl, a cyano group, an aldehyde group, a ketone group, a carboxyl group, an ester group, a carbamoyl group, an amino group, a nitro group or a silyl group.

As described above, the aforementioned radicals may be substituted by E and/or, if desired, interrupted by D. Interruptions are of course possible only in the case of radicals containing at least 2 carbon atoms connected to one another by single bonds; C₆-C₁₈aryl is not interrupted; interrupted arylalkyl or alkylaryl contains the unit D in the alkyl moiety. C₁-C₁₈alkyl substituted by one or more E and/or interrupted by one or more units D is, for example, (CH₂CH₂O)ₙ—R^{x}, where n is a number from the range 1-9 and R^{x} is H or C₁-C₁₀alkyl or C₂-C₁₀alkanoyl (e.g. CO—CH(C₂H₅)C₄H₉), CH₂—CH(OR^{y′})—CH₂—O—R^{y}, where R^{y} is C₁-C₁₈alkyl, C₅-C₁₂cycloalkyl, phenyl, C₇-C₁₅phenylalkyl, and R^{y′} embraces the same definitions as R^{y} or is H; C₁-C₈alkylene-COO—R^{z}, e.g. CH₂COOR_{z}, CH(CH₃)COOR^{z}, C(CH₃)₂COOR^{z}, where R^{z} is H, C₁-C₁₈alkyl, (CH₂CH₂O)₁₋₉—R^{x}, and R^{x} embraces the definitions indicated above; CH₂CH₂—O—CO—CH═CH₂; CH₂CH(OH)CH₂—O—CO—C(CH₃)═CH₂.

The electroluminescent devices may be employed for full color display panels in, for example, mobile phones, televisions and personal computer screens.

The following Examples illustrate the invention. In the Examples and throughout this application, the term light emitting material means the present triazine compounds.

### EXAMPLES

### Example 1

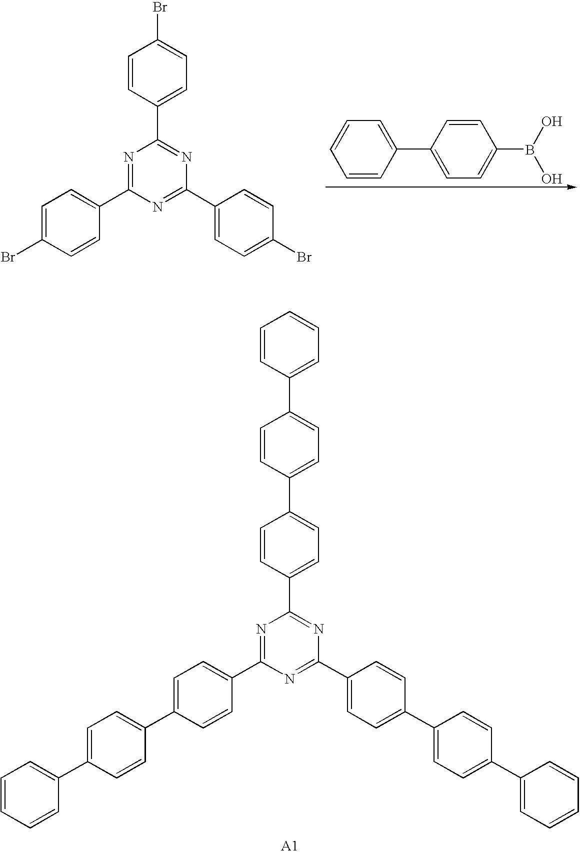

In a 25 ml three necked vessel equipped with a reflux condenser, an argon inlet and a thermometer, 0.5 g 2,4,6-tris-(4-bromophenyl)-1,3,5-triazine [synthesized according to Hayami S., Inoue K., Chem. Letters, (1999), (7), 545-546] and 0.816 g 4-biphenylboronic acid are added to 10 ml of toluene under argon. Then a solution of 2.238 g CsCO₃ in 3.5 ml water is added, and 1% of a palladium(II)catalyst [described in W099/47474] is added. Then the mixture is heated to reflux for 3 hours. The product is filtered and washed with acetone. Then the product is recrystallized from dimethylformamide. The fine crystals are washed with isopropanol and dried. 0.51 g of pure product (A1) are obtained. Melting point: 360° C.

### ¹H NMR (CDCl₃, 300 MHz): δ=8.91 (6H), 7.89 (6H), 7.82 (6H), 7.75 (6H), 7.68 (6H), 7.49 (6H), 7.40 (3H).

### Example 2

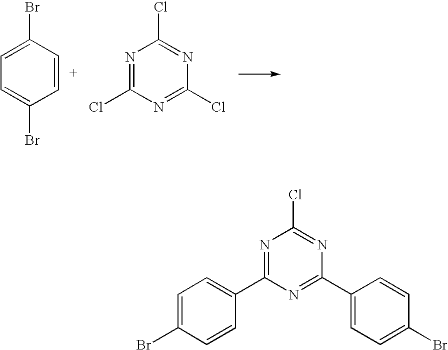

A solution of 28.3 g (0.120 mol) p-dibromo-benzene in 90 ml terahydrofurane (THF) is added dropwise under nitrogen to 3.21 g (0.132 mol) of magnesium in 10 ml diethyl ether. The p-dibromo-benzene solution is added in a way that the reaction mixture keeps refluxing. After the addition of the p-dibromo-benzene solution the reaction mixture is stirred for 1 h. This solution is then added to a solution of 5.53 g (30 mmol) of cyanuric chloride in 50 ml THF. The reaction mixture is stirred for 5 h at 20° C. and then hydrolysed with water and 20% hydrochloric acid. The water phase is extracted with dichloromethane. The organic phase is dried with magnesium sulfate and filtered on silica gel with dichloromethane. The solvent is removed in vacuum. After recrystallisation in toluene 2.1 g (4.93 mmol; yield 16%) of the product are obtained.

### Example 3

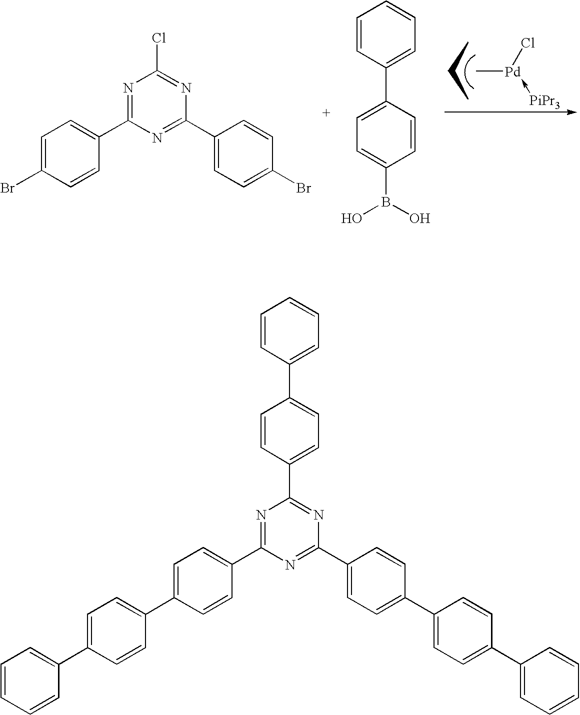

1.72 g (8.75 mmol) phenylbronic acid are added to 1.06 g (2.5 mmol) 1,3-bis-(-p-bromophenyl)-5-chloro-triazine in 50 ml dimethoxyethane (DME) under argon. To this solution 2.85 g (8.75 mmol) Cs₂CO₃ in 5 ml water and 1% of a palladium(II)catalyst [described in W099/47474] are added. The reaction mixture is refluxed for 18 h and then diluted with water. The product is filtered off, washed with 20% hydrochloric acid and then water. After recrystallisation in DMF 1.1 g (1.59 mmol; yield 64%) of the product are obtained.

### Application Example 1

Present compound A1, as light emitting material, 2,5-bis(1-naphthyl)-1,3,4-oxadiazole and a polycarbonate resin in a weight ratio of 5:3:2 are dissolved in tetrahydrofuran, and the solution is spin-coated on a cleaned glass substrate with an ITO electrode to form a light-emitting layer having a thickness of 100 nm. An electrode having a thickness of 150 nm is formed thereon from a magnesium/indium alloy having a magnesium/indium mixing ratio of 10/1, to obtain an organic EL device. The device exhibits light emission with excellent brightness and efficiency at a direct current voltage of 5 V.

### Application Example 2

Present compound A1 is vacuum-deposited on a cleaned glass substrate with an ITO electrode to form a light-emitting layer having a thickness of 100 nm. An electrode having a thickness of 100 nm is formed thereon from a magnesium/silver alloy having a magnesium/silver mixing ratio of 10/1, to obtain an organic EL device. The light-emitting layer is formed by deposition under a vacuum of 10⁻⁶ Torr at a substrate temperature of room temperature. The device shows emission having an excellent brightness and efficiency at a direct current voltage of 5 V.

### Application Example 3

Present compound A1 is dissolved in methylene chloride tetrahydrofuran, and the solution is spin-coated on a cleaned glass substrate with an ITO electrode to form a light-emitting layer having a thickness of 50 nm. Then, aluminum bis(2-methyl-8-quinolinate)(2-naphtolate) is vacuum-deposited to form an electron transporting layer having a thickness of 10 nm, and an electrode having a thickness of 100 nm is formed thereon from a magnesium/aluminum alloy having a magnesium/aluminum mixing ratio of 10/1, to obtain an organic EL device. The light-emitting layer and the electron-injecting layer are formed by deposition under a vacuum of 10⁻⁶ Torr at a substrate temperature of room temperature. The device shows an emission having an excellent brightness and efficiency at a direct current voltage of 5 V.

### Application Example 4

One of hole transporting materials (H-1) to (H-6) is vacuum-deposited on a cleaned glass substrate with an ITO electrode, to form a hole transporting layer having a thickness of 30 nm. Then, present compound A1 is vacuum-deposited to form a light-emitting layer having a thickness of 30 nm. Further, one of electron transporting materials (E-1) to (E-6) is vacuum-deposited to form an electron transporting layer having a thickness of 30 nm. An electrode having a thickness of 150 nm is formed thereon from a magnesium/silver alloy having a magnesium/silver mixing ratio of 10/1, to obtain an organic EL device. Each layer is formed under a vacuum of 10⁶ Torr at a substrate temperature of room temperature. All the organic EL devices obtained in these Examples shows high brightness and efficiency.

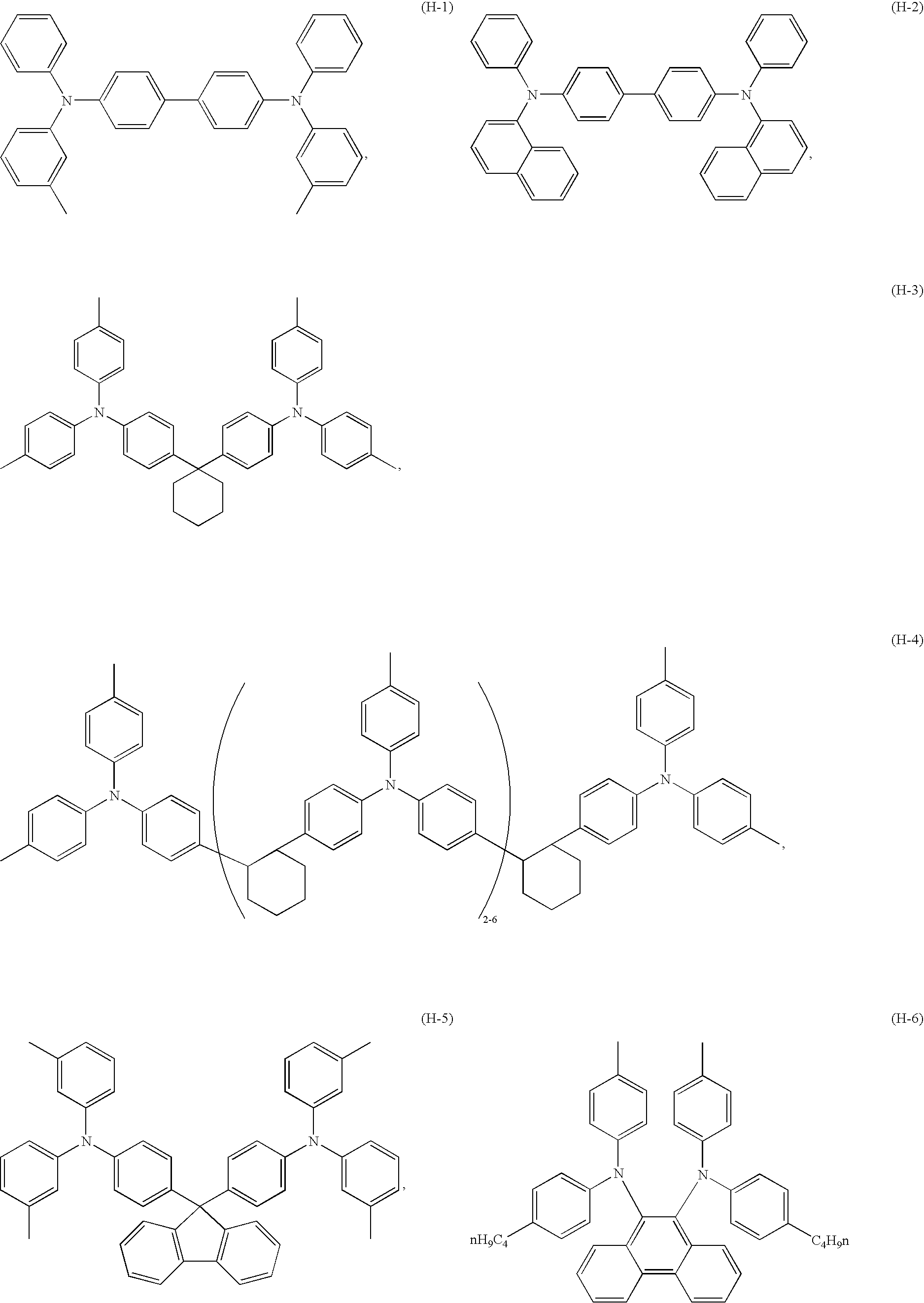

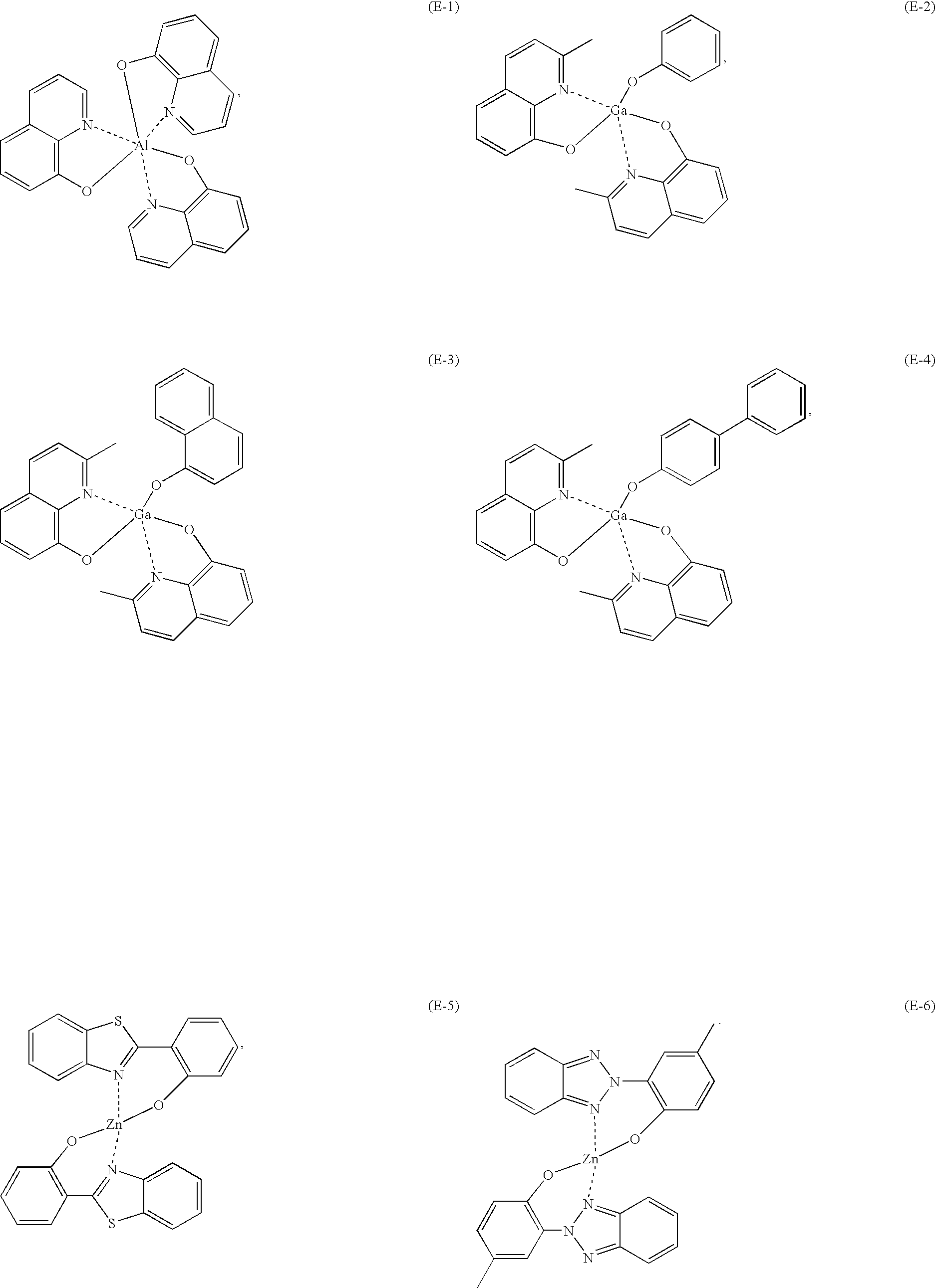

### Application Example 5

On a cleaned glass substrate with an ITO electrode, 4,4′,4″-tris(N-(3-methylphenyl)-N-phenylamino)triphenylamine is vacuum-deposited to form a hole-injecting layer having a thickness of 25 nm. Further, a hole transporting material (H-1) is vacuum-deposited to form a hole transporting layer having a thickness of 5 nm. Then, compound A1 as light-emitting material is vacuum-deposited to form a light-emitting layer having a thickness of 20 nm. Further, an electron transporting material (E-1) is vacuum-deposited to form an electron transporting layer having a thickness of 30 nm. Then, an electrode having a thickness of 150 nm is formed thereon from a magnesium/silver alloy having an magnesium/silver mixing ratio of 10/1, to obtain an organic EL device. The device shows emission having an outstanding brightness and efficiency at a direct current voltage of 5 V.

### Application Example 6

A hole transporting material (H-5) is vacuum-deposited on a cleaned glass substrate with an ITO electrode to form a hole transporting layer having a thickness of 20 nm. Then, compound A1 as light-emitting material is vacuum-deposited to form a light-emitting layer having a thickness of 20 nm. Further, an electron transporting material (E-2) is vacuum-deposited to form a first electron transporting layer having a thickness of 20 nm. Then, an electron transporting material (E-5) is vacuum-deposited to form a second electron transporting layer having a thickness of 10 nm, and an electrode having a thickness of 150 nm is formed thereon from a magnesium/silver alloy having an magnesium/silver mixing ratio of 10/1, to obtain an organic EL device. The device shows light emission having an excellent brightness and efficiency at a direct current voltage of 5 V.

### Application Example 7

An organic EL device is prepared in the same manner as in Example 4 except that the light-emitting layer is replaced with a 30 nm thick light-emitting layer formed by vacuum-depositing compound Al and one of the dopant compounds (D-1) to (D-7) in a weight ratio of 100:1. All the organic EL devices obtained in these Examples shows high brightness characteristics and gives intended light emission colors.

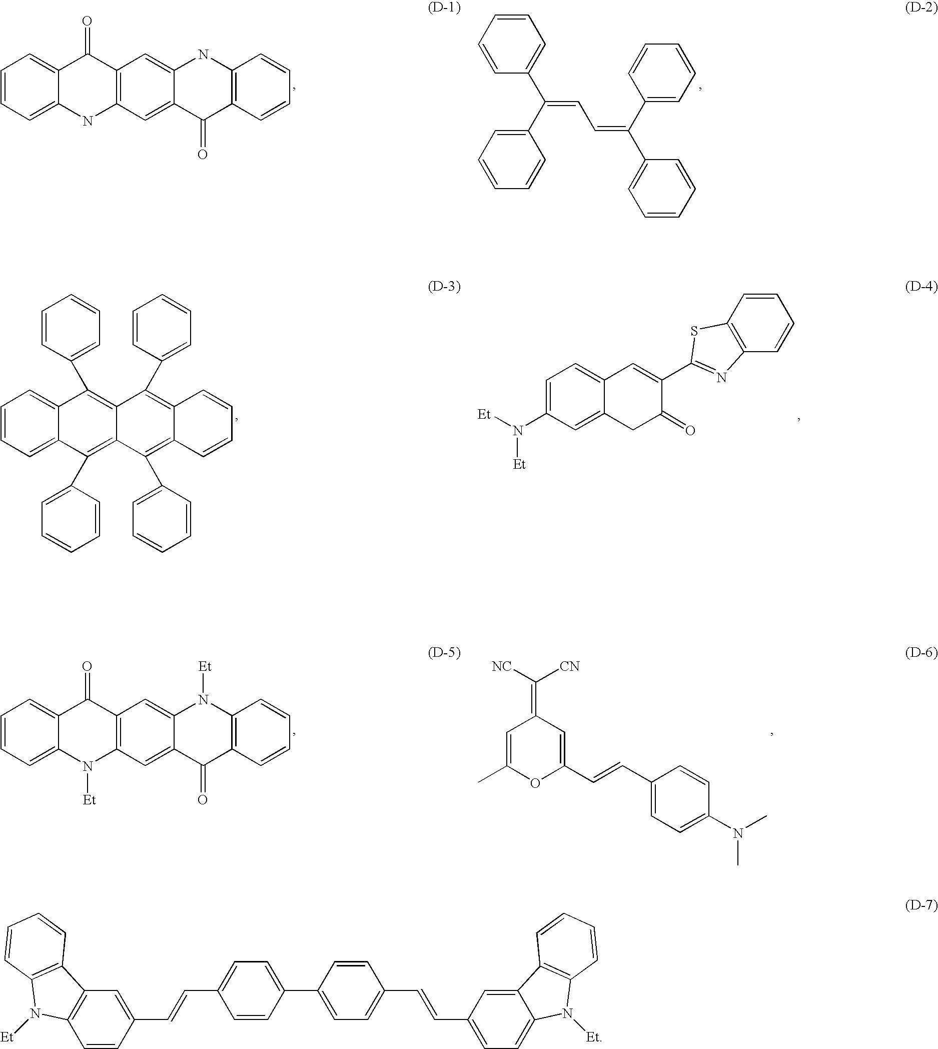

### Application Example 8

On a cleaned glass substrate with an ITO electrode, N,N′-1-naphthyl-N,N′-diphenyl-1,1′-biphenyl-4,4′-diamine and 5,10-diphenylanthracene are vacuum-deposited to form a hole-injecting layer. Further, 4,4′-bis(9-carbazolyl)-1,1′-biphenyl is vacuum-deposited to form a hole transporting layer. Then, compound A1 as light-emitting material is vacuum-deposited to form a light-emitting layer. Then, an electrode is formed thereon from a magnesium/silver alloy having an magnesium/silver mixing ratio of 9/1, to obtain an organic EL device. The device shows emission having an outstanding brightness and efficiency at a direct current voltage of 5 V.

The organic EL devices obtained in the Application Examples of the present invention show an excellent light emission brightness and achieved a high light emission efficiency. When the organic EL devices obtained in the above Examples are allowed to continuously emit light at 3 (mA/cm²), all the organic EL devices remain stable. Since the light-emitting materials of the present invention have a very high fluorescence quantum efficiency, the organic EL devices using the light-emitting materials achieved light emission with a high brightness in a low electric current applied region, and when the light-emitting layer additionally uses a doping material, the organic EL devices are improved in maximum light emission brightness and maximum light emission efficiency. Further, by adding a doping material having a different fluorescent color to the light-emitting material of the present invention, there are obtained light-emitting devices having a different light emission color. The organic EL devices of the present invention accomplish improvements in light emission efficiency and light emission brightness and a longer device life, and does not impose any limitations on a light-emitting material, a dopant, a hole transporting material, an electron transporting material, a sensitizer, a resin and an electrode material used in combination and the method of producing the device. The organic EL device using the material of the present invention as a light-emitting material achieves light emission having a high brightness with a high light emission efficiency and a longer life as compared with conventional devices. According to the light-emitting material of the present invention and the organic EL device of the present invention, there can be achieved an organic EL device having a high brightness, a high light emission efficiency and a long life.

1. A triazine compound of formula I 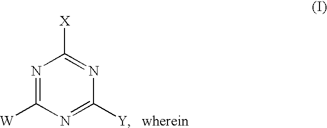
W, X and Y are independently of each other a group of formula 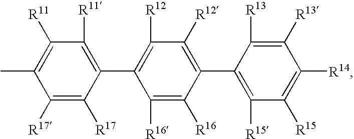
wherein
R¹¹, R^{11′}, R¹², R^{12′}, R¹³, R^{13′}, R¹⁵, R^{15′}, R¹⁶, R^{16′}, R¹⁷, and R^{17′} are independently of each other H, E, C₆-C₁₈aryl; C₆-C₁₈aryl which is substituted by G; C₁-C₁₈alkyl; C₁-C₁₈alkyl which is substituted by E and/or interrupted by D; C₇-C₁₈aralkyl; or C₇-C₁₈aralkyl which is substituted by G; R
or
R^{11′} and R¹², R^{12′} and R¹³, R^{15′} and R¹⁶, and/or R^{16′} and R¹⁷, are each a divalent group L¹ selected from an oxygen atom, an sulfur atom, >CR¹⁸R¹⁹>SiR¹⁸R¹⁹, or 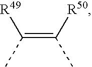
wherein
R¹⁸ and R¹⁹ are independently of each other C₁-C₁₈alkyl; C₁-C₁₈alkoxy, C₆-C₁₈aryl; C₇-C₁₈aralkyl;
R¹¹ and R^{11′}, R¹² and R^{12′}, R¹³ and R^{13′}, R^{13′} and R¹⁴, R2 ¹⁴ and R¹⁵, R¹⁵ and R^{5′}, R¹⁶ and
R^{16′}, and/or R^{17′} and R¹⁷, are each a divalent group 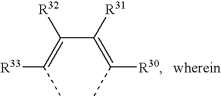
R³⁰, R³¹, R³², R³³, R⁴⁹ and R⁵⁰ are independently of each other H, C₁-C₁₈alkyl; C₁-C₁₈alkyl, which is substituted by E and/or interrupted by D; E; C₆-C₁₈aryl; C₆-C₁₈aryl, which is substituted by E;
and
R¹⁴ is H, E, C₂-C₃₀heteroaryl, C₂-C₃₀heteroaryl, which is substituted by G, C₆-C₃₀aryl, or C₆-C₃₀aryl, which is substituted by G, C₁-C₁₈alkyl; or C₁-C₁₈alkyl which is substituted by E and/or interrupted by D;
D is —CO—; —COO—; —OCOO—; —S—; —SO—; —SO₂—; —O—; —NR⁵—; SiR⁶¹R⁶²—; —POR⁵—; —CR⁶³═CR⁶⁴—; or —C═C—;
E is —OR⁵; —SR⁵; —NR⁵R⁶; —COR⁸; —COOR⁷; —OCOOR⁷, —CONR⁵R⁶; —CN; or halogen; G is E, or C₁-C₁₈alkyl,
wherein R⁵ and R⁶ are independently of each other C₆-C₁₈aryl; C₆-C₁₈aryl which is substituted by C₁-C₁₈alkyl, C₁-C₁₈alkoxy; or C₁-C₁₈alkyl which is interrupted by —O—; or R⁵ and R⁶together form a five or six membered ring,
R⁷ is C₆-C₁₈aryl; C₆-C₁₈aryl which is substituted by C₁-C₁₈alkyl, C₁-C₁₈alkoxy; or C₁-C₁₈alkyl which is interrupted by —O—;
R⁸ is C₇-C₁₂alkylaryl; C₁-C₁₈alkyl; or C₁-C₁₈alkyl which is interrupted by —O—;
R⁶¹ and R⁶² are independently of each other C₆-C₁₈aryl; C₆-C₁₈aryl which is substituted by C₁-C₁₈alkyl, C₁-C₁₈alkoxy; or C₁-C₁₈alkyl which is interrupted by —O—, and
R⁶³ and R⁶⁴ are independently of each other H, C₆-C₁₈aryl; C₆-C₁₈aryl which is substituted by C₁-C₁₈alkyl, C₁-C₁₈alkoxy; or C₁-C₁₈alkyl which is interrupted by —O—.

2. A triazine compound of formula I according to claim 1, wherein W, X and Y are independently of each other a group of formula 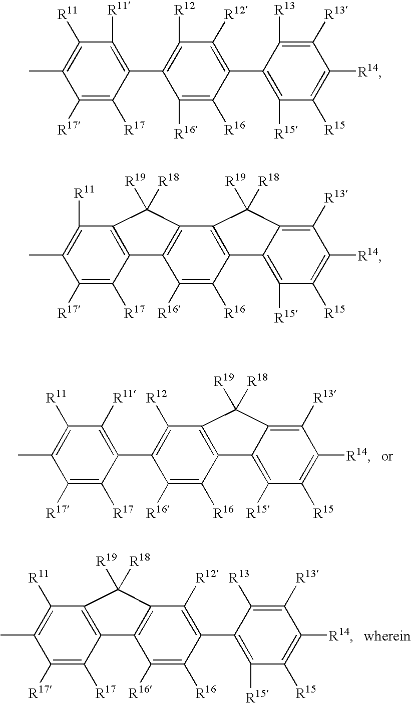
R¹¹, R^{11′}, R¹², R^{12′}, R¹³, R^{13′}, R¹⁵, R^{15′}, R¹⁶, R^{16′}, R¹⁷ and R^{17′} are independently of each other H, C₆-C₁₈aryl; C₆-C₁₈aryl which is substituted by G; E, C₁-C₁₈alkyl; C₁-C₁₈alkyl which is substituted by E and/or interrupted by D; C₇-C₁₈aralkyl; C₇-C₁₈aralkyl which is substituted by G; and
D, E, G, R¹⁴, R¹⁸ and R¹⁹ are as defined in claim 1, or
W is a group of the formula —W¹—W²—W³,
X is a group of the formula —X¹—X²—X³ and
Y is a group of the formula —Y¹—Y²—Y³, wherein W¹, W², X¹, X², Y¹ and Y² are independently of each other a group of formula 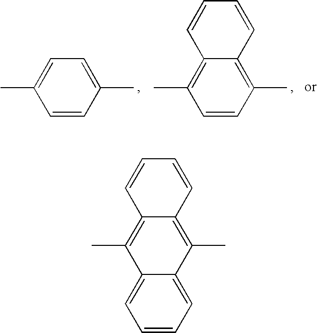
and W³, X³ and Y³ are independently of each other a group of formula 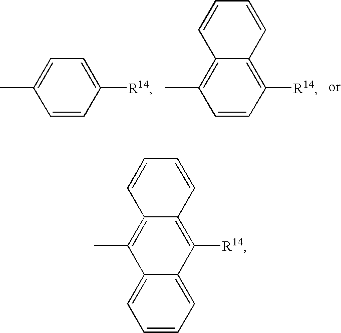
wherein R¹⁴ is as defined above.

3. The triazine compound according to claim 1, wherein R¹¹, R^{11′}, R¹², R^{12′}, R¹³, R^{13′}, R¹⁵, R^{15′},
R¹⁶, R^{16′}, R¹⁷ and R^{17′} as well as R¹⁴, are independently of each other H, E; or C₁-C₈alkyl; wherein E is —OR⁵; —SR⁵; —NR⁵R⁶; —COR⁸; —COOR⁷; —CONR⁵R⁶; —CN; —OCOOR⁷; or halogen; wherein R⁵ and R⁶ are independently of each other C₆-C₁₂aryl, or C₁-C₈alkyl;
R⁷ is C₇-C₁₂alkylaryl, or C₁-C₈alkyl; and
R⁸ is C₆-C₁₂aryl; or C₁-C₈alkyl.

4. The triazine compound according to claim 1, wherein
W, X and Y are a group of formula 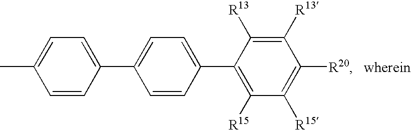
R¹³, R^{13′}, R¹⁵ and R^{15′} are H and R²⁰ is H, 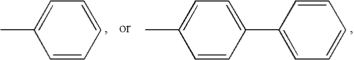
or R¹³ and
R¹⁵ are H, R^{13′} and R^{15′} are independently of each other H, C₁-C₈alkyl, or C₁-C₈alkoxy, and R²⁰ is H, C₁-C₈alkyl, or C₁-C₈alkoxy; or
R¹³, R¹⁵ and R^{15′} are H, and R^{13′} and R²⁰ are 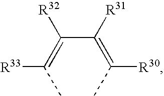
R²⁰, R¹⁵ and R^{15′} are H, and R¹³ and R^{13′} are 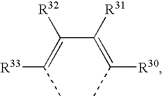
wherein R³⁰, R³¹ , R³² and R³³ are H, C₁-C₈alkyl, or C₁-C₈alkoxy.

5. A triazine compound according to claim 1, wherein
W, X and Y are independently of each other a group of formula 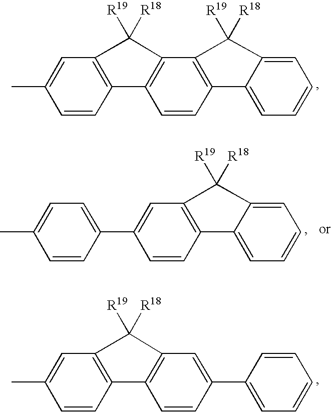
wherein R¹⁸ and R¹⁹ are independently of each other C₁-C₈alkyl.

6. An electroluminescent device, comprising a triazine compound of formula I according to claim 1.

7. Electroluminescent device according to claim 6, wherein the electroluminescent device comprises in this order
(a) an anode
(b) a hole injecting layer and/or a hole transporting layer
(c) a light-emitting layer
(d) optionally an electron transporting layer and
(e) a cathode.

8. Electroluminescent device according to claim 7, wherein the triazine compound of formula I forms the light-emitting layer.

9. Electrophotographic photoreceptors, photoelectric converters, solar cells, image sensors, dye lasers and electroluminescent devices comprising a triazine compound according to claim 1.

10. A triazine compound according to claim 1, wherein R⁵ and R⁶ together form

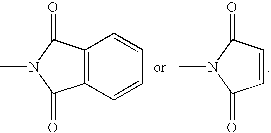

11. A triazine compound of formula I according to claim 2, wherein
W is a group of the formula —W¹—W²—W³,
X is a group of the formula —X¹—X²—X³ and
Y is a group of the formula —Y¹—Y²—Y^{3,} wherein W¹, W², X¹, X², Y¹ and Y² are independently of each other a group of formula 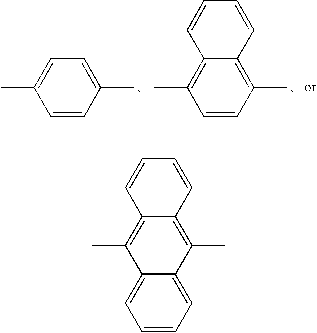
and W³, X³ and Y³ are independently of each other a group of formula 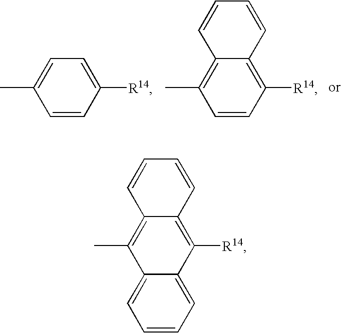
wherein R¹⁴ is —SR⁵; —NR⁵R⁶; —COR⁸; —COOR⁷; —OCOOR⁷, —CONR⁵R⁶; —CN, C₆-C₃₀aryl, or C₆-C₃₀aryl, which is substituted by G, or C₁-C₁₈alkyl which is substituted by E and/or interrupted by D.

12. The triazine compound according to claim 1, wherein R¹¹, R^{11′}, R¹², R^{12′}, R¹³, R^{13′}, R¹⁵, R^{15′}, R¹⁶, R^{16′}, R¹⁷ and R^{17′} as well as R¹⁴, are independently of each other H, E; or C₁-C₈alkyl; wherein E is —SR⁵; —NR⁵R⁶; —COR⁸; —COOR⁷; —CONR⁵R⁶; —CN; —OCOOR⁷; wherein R⁵ and R⁶ are independently of each other C₆-C₁₂aryl, or C₁-C₈alkyl;
R⁷ is C₇-C₁₂alkylaryl, or C₁-C₈alkyl; and
R⁸ is C₆-C₁₂aryl; or C₁-C₈alkyl.

13. A triazine compound according to claim 1, which is a compound of formula: 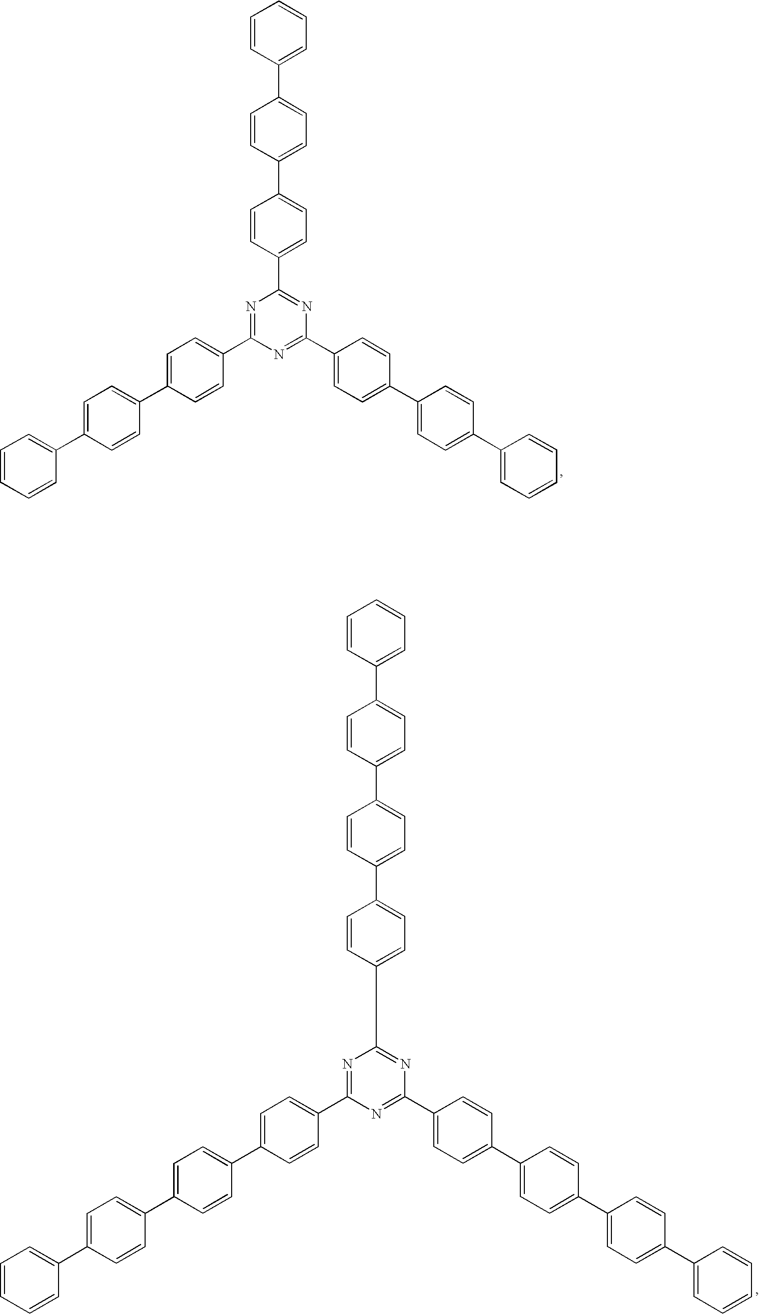 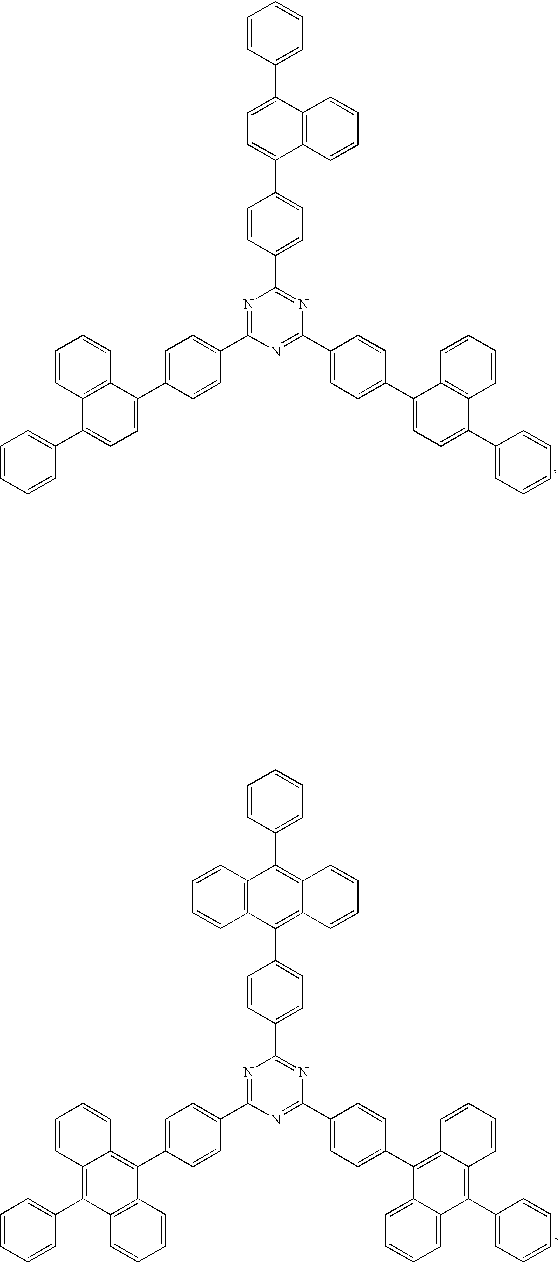 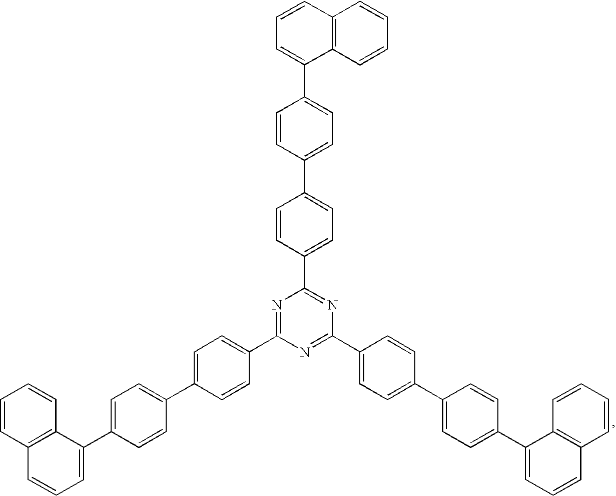 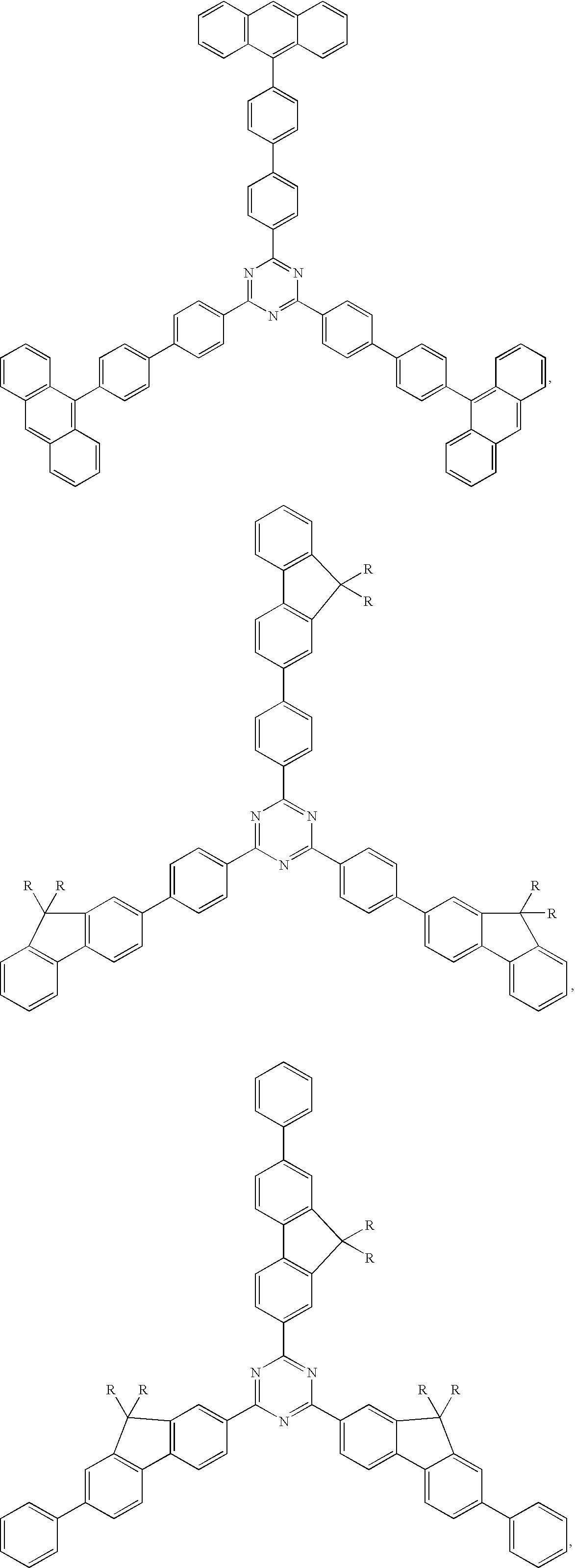 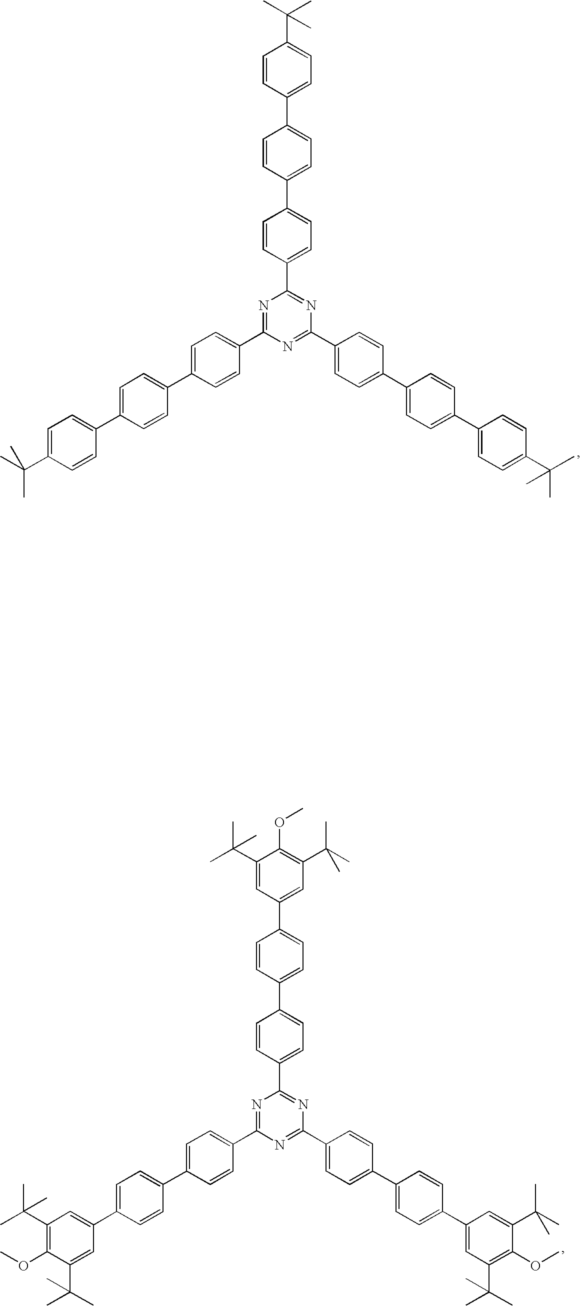 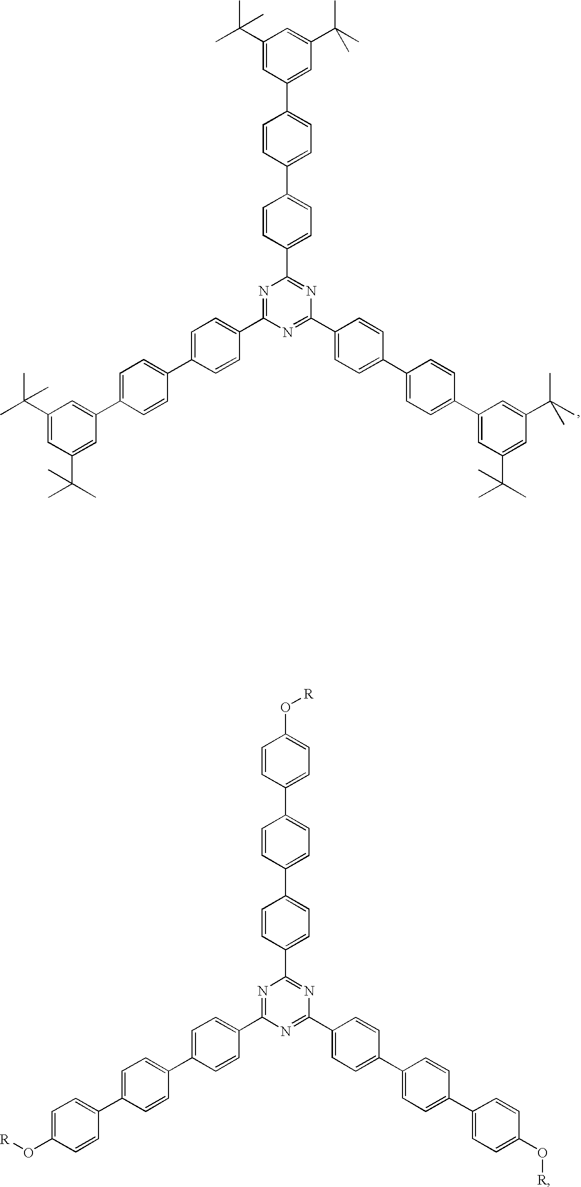 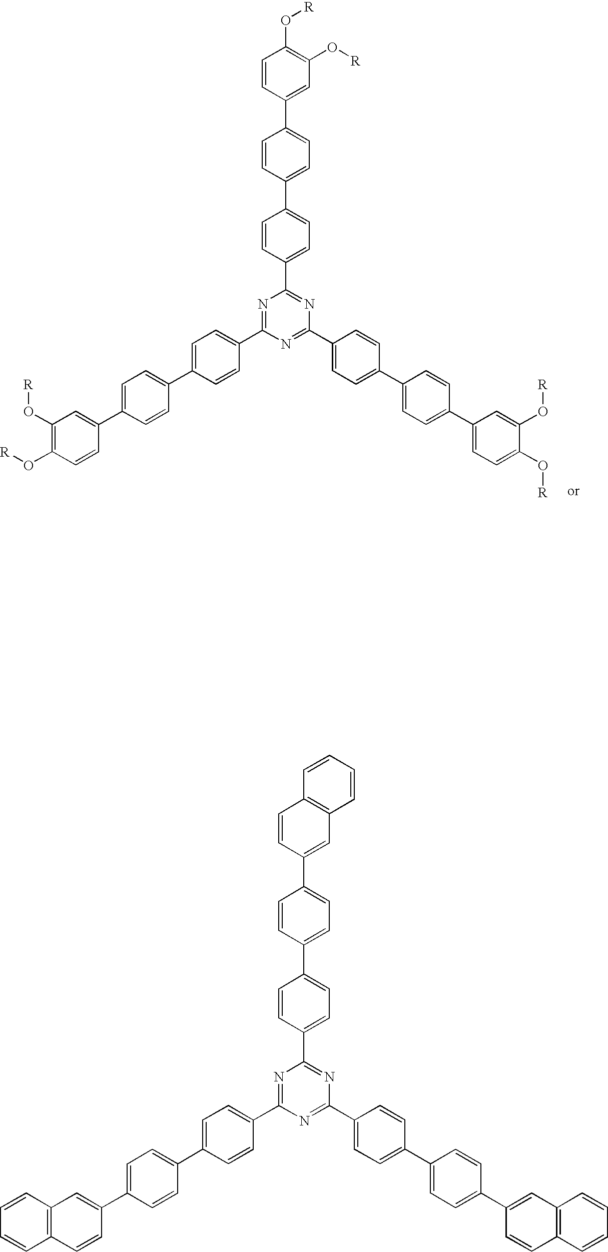
wherein R is methyl, ethyl, n-butyl, iso-butyl or n-octyl.

14. A triazine compound according to claim 1, which is a compound of formula: 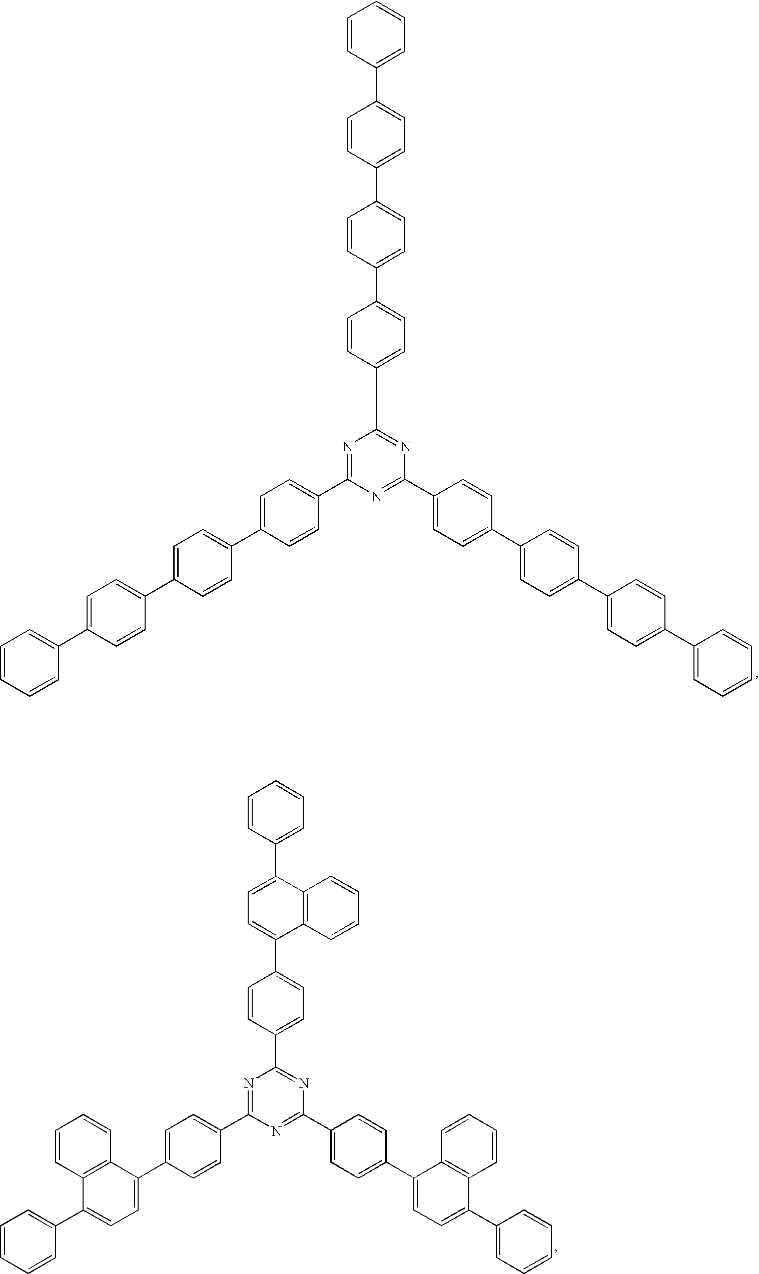 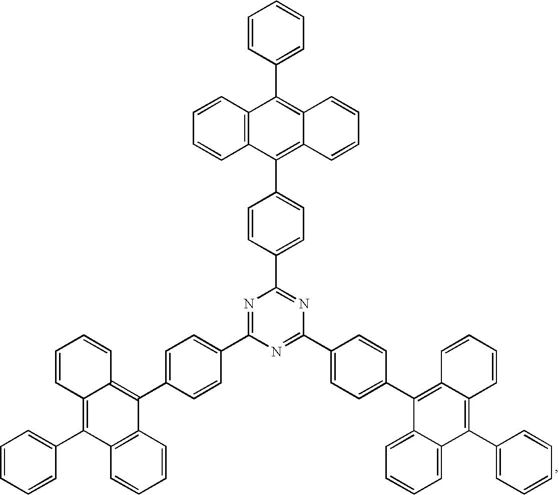 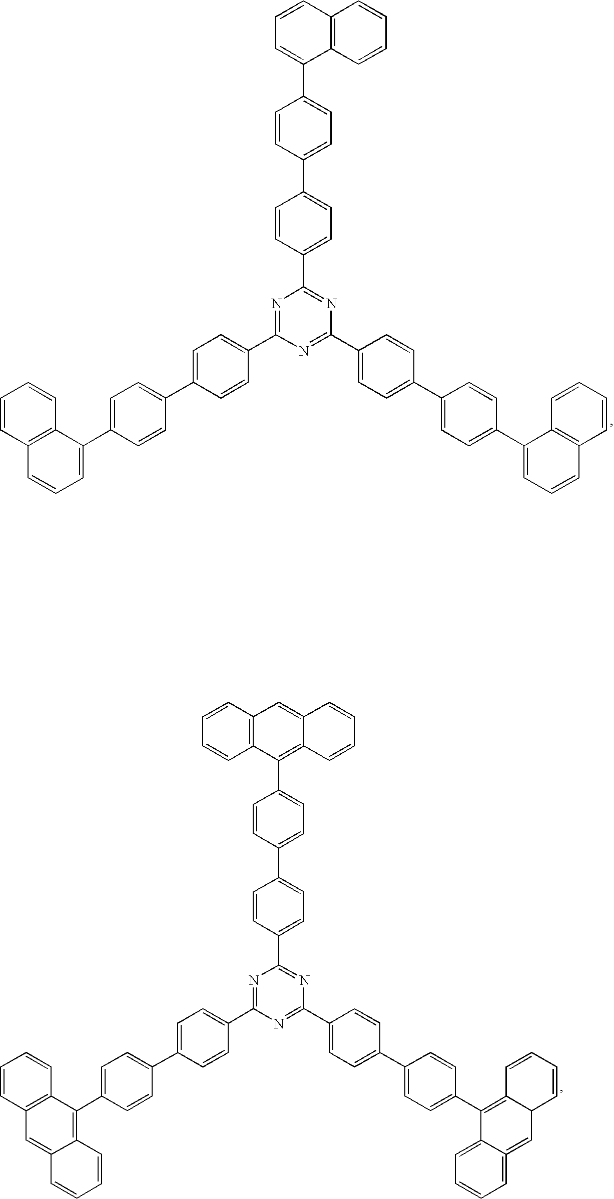 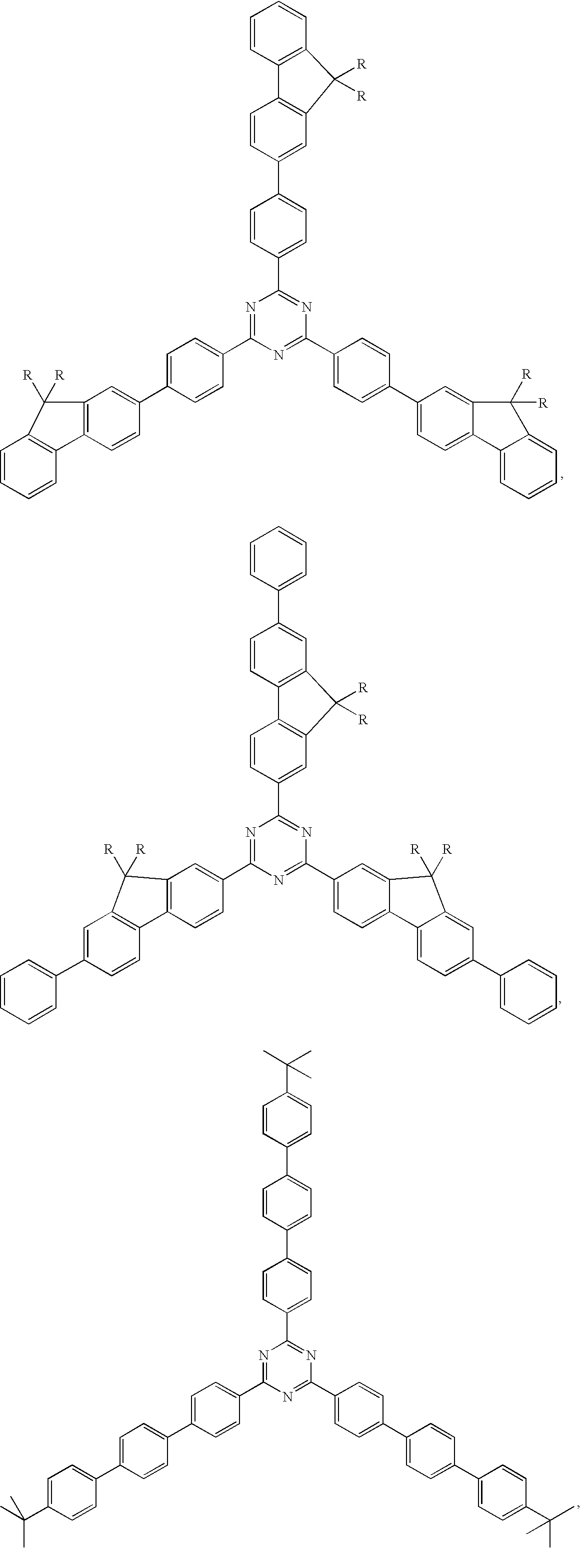 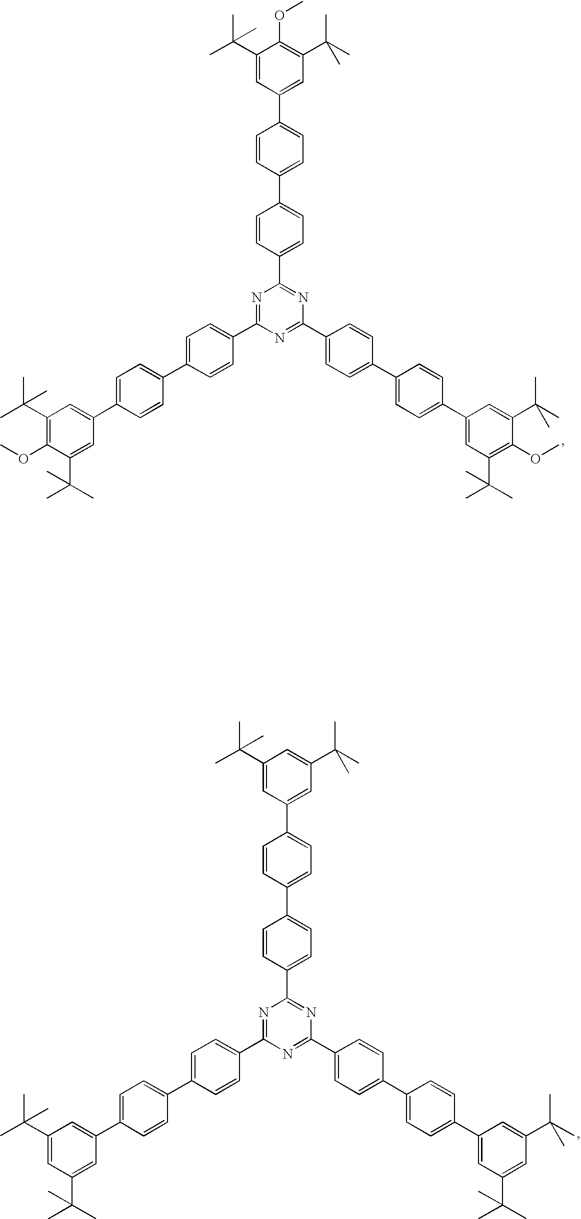 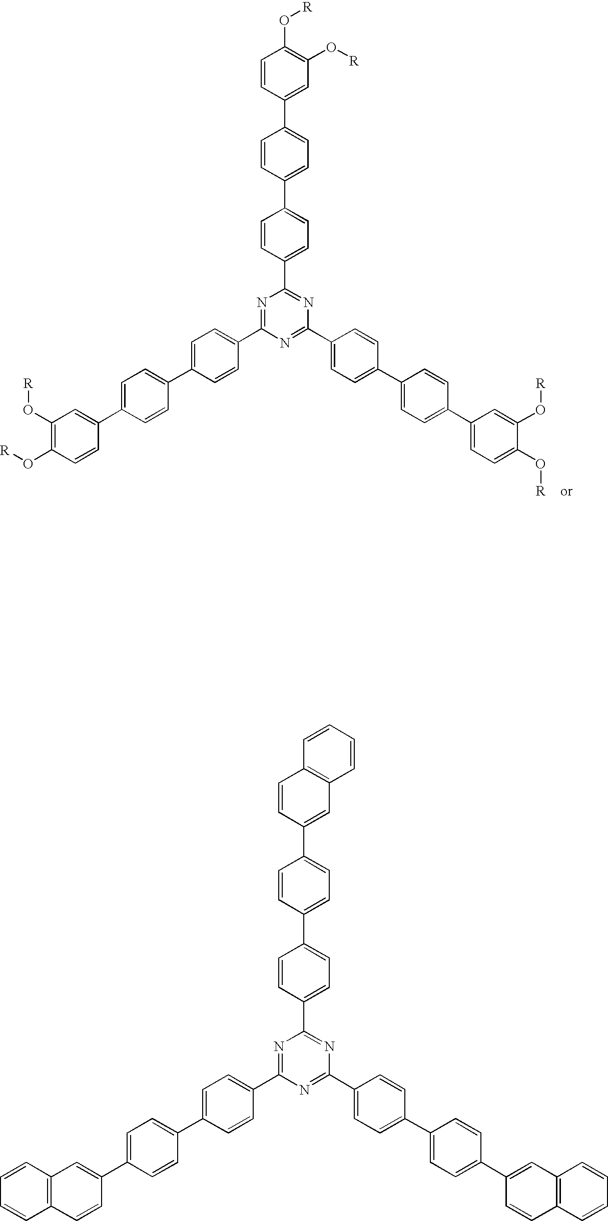
wherein R is methyl, ethyl, n-butyl, iso-butyl or n-octyl.
